(19) United States
(12) Patent Application Publication
(10) Pub. No.: US 2023/0024271 A1
(43) Pub. Date: Jan. 26, 2023

(54) MODULAR VAPORIZER SYSTEM AND METHOD FOR VAPORIZING A COMPOSITION

(71) Applicant: Hauni Maschinenbau GmbH, Hamburg (DE)

(72) Inventors: Tobias Wuttke, Reinbek (DE); Lasse Cornils, Hamburg (DE); Christian Hanneken, Hamburg (DE); Michael Kleine Wächter, Lankau (DE); Niklas Romming, Hamburg (DE); Björn Schlüter, Ahrensburg (DE); Tim Ullner, Hamburg (DE); Volkmar Voigtländer, Hamburg (DE)

(21) Appl. No.: 17/786,341

(22) PCT Filed: Dec. 15, 2020
(86) PCT No.: PCT/EP2020/086124

§ 371 Date: Jun. 16, 2022

(30) Foreign Application Priority Data
Dec. 19, 2019 (DE) 10 2019 135 176.6

(51) Int. Cl. A24F 40/465; A24F 40/10; A24F 40/42; A24F 40/485

(52) U.S. Cl. CPC A24F 40/465; A24F 40/10; A24F 40/42; A24F 40/485

(57) ABSTRACT

A vaporiser system for vaporising a composition includes a first element with at least one radiation source connected to an electrical energy source which is adapted to emit electromagnetic radiation, and a second element with at least one reservoir for holding the composition and at least one absorber. The first and the second element are reversibly and detachably connectable to each other in a non-destructive manner. A radiation conductor is arranged such that a radiation-conducting connection is formed between the radiation source and the absorber when the first element and the second element are connected to each other. The vaporiser system is adapted to vaporise the composition by the thermal energy obtained from the electromagnetic radiation by the absorber via conversion and/or by the electromagnetic radiation with increased wavelength compared to the absorbed electromagnetic radiation which is emitted by the absorber.

### CROSS-REFERENCE TO RELATED APPLICATIONS

This application is the U.S. National Stage of PCT/EP2020/086124 filed Dec. 15, 2020, which claims priority to German Patent Application No. 10 2019 135 176.6, filed Dec. 19, 2019, the content of both of which are incorporated herein by reference in their entirety.

### FIELD OF THE INVENTION

The invention relates to a vaporiser system for vaporising a composition, a cartridge for a corresponding vaporiser system, a portable vaporising apparatus comprising a corresponding vaporiser system, an absorber for a corresponding vaporiser system, a composition for a corresponding vaporiser system, a spatial juxtaposition of a plurality of components of a corresponding vaporiser system, and a method for vaporising a composition in a vaporiser system. Also disclosed are uses of corresponding cartridges, absorbers and compositions in corresponding vaporiser systems.

### BACKGROUND OF THE INVENTION

It has been well-known for centuries that administering active ingredients via the respiratory tract is an efficient and gentle method of supplying the human or animal body with physiologically active ingredients, traditional inhalation methods in particular, which can be carried out with sometimes the simplest means, having found a firm place both in conventional medicine and among home remedies. In these simple methods, an active ingredient dissolved in a carrier substance, frequently water, is usually heated in a pot or comparable vessel and thereby caused to vaporise.

Due to the increasingly critical estimation of smoking in many parts of the world, i.e. the consumption of tobacco products by burning them and inhaling the resulting smoke, for example in the form of cigarettes or cigars, the focus of interest in recent years has increasingly been on inhalation methods in which the physiologically active ingredients that are traditionally absorbed via tobacco smoke are instead applied via corresponding inhalation methods that do not involve burning tobacco, this concept also being applied to other active ingredients that are otherwise frequently associated with smoking, such as tetrahydrocannabinol (THC) and other cannabinoids.

Progressive technical development has made it possible to design ever smaller corresponding vaporiser systems for vaporising a composition containing active ingredients so that today vaporiser systems are available with which vaporising a composition containing active ingredients can take place in a portable hand-held device which, for example, can be the size of a traditional cigar or a packet of cigarettes. The most prominent uses for corresponding vaporiser systems are electronic cigarettes and inhalers for medical applications.

The systems known today are mostly based on the fact that a composition stored in a reservoir, which is regularly referred to as liquid, is vaporised by a more or less controlled supply of thermal energy from a heating element, e.g. a coiled filament, so that the user can inhale the resulting vapours. The liquid from the reservoir to the heating element is often supplied by a wick, so that reference is frequently made to wick/coil systems. A corresponding system is disclosed, for example, in US 20140096782 A1.

In recent years, some experts have come to realise that these wick/coil systems are often disadvantageous as they are often perceived as too uncontrolled and too inefficient. For example, the arrangement of wick and heater varies greatly in some cases due to the production process, with the result that a different quantity of liquid and thus also of active ingredient is vaporised per puff depending on the production example. In addition, areas frequently occur on the heater where no liquid is available. Furthermore, there are often weak spots in the heating wire or structural defects in the heating grid where unintentionally high heating occurs, resulting in harmful decomposition products. Accordingly, in recent years new vaporiser systems have been developed which eliminate or reduce the disadvantages known for wick/coil systems. Corresponding systems are disclosed, for example in DE102017111435, although it has been shown that in many cases many customary concepts are not easily transferable from wick/coil concepts to more modern concepts.

In light of the increasing awareness of the population regarding sustainability and resource-conserving handling of valuable recyclable materials, there is a steadily growing interest in developing appropriate vaporiser systems which generate as little waste as possible, with vaporiser systems with refillable reservoirs being particularly widespread on the market. Refillable systems, however, are regularly viewed as disadvantageous for many reasons. In particular, refilling by users is often complicated and brings them into contact with the liquid containing the active ingredient which can also contaminate it. Due to the possibility of the user, consciously or unconsciously, filling the reservoir with unauthorised substances and vaporising them, there is not only a risk to the health of the user but the operational reliability and durability of the device may also be adversely reduced.

In prior art, the concept described above, which is advantageous per se, has been realised mainly with electric heating elements so far. This means that electrical energy is supplied from the reusable part to the heater arranged in the disposable part. This established arrangement, however, has considerable disadvantages from the point of view of the person skilled in the art. In this case, it is absolutely essential to provide reliable and mechanically resilient electrical contacting between the cartridge and the reusable part to guarantee the power supply to the heating element in the cartridge even if it is replaced multiple times. This requires technically complex and therefore cost-intensive electrical contacting which will nevertheless always represent a weak spot in the system and places high demands on production operations. Beyond this, the configuration of the heating elements and cartridges, as well as their arrangement in a vaporiser system, is severely limited in customary systems by the absolutely essential electrical contacting of the heating element. In addition, these systems, which require a physical connection between the reusable part and the heating element, are known to often have problems with the leak tightness of the system, as the heating element is in turn in contact with the liquid so that relatively complex and expensive seals may be necessary. In these systems, in terms of cost and effort for the seal, it is effectively not economically feasible to provide the heating element in the reusable part. However, the heating element is a comparatively complex and costly component to produce, and in this case, it can only be disposed of with the cartridge after use which is disadvantageous from an economic and/or environmental point of view. In addition, the heating element and the electronic contacting regularly consist of other materials (frequently metals, semiconductors or ceramics) than the reservoir (frequently glass or plastic) and, if applicable, the jacket of the cartridge (frequently plastic) so that recycling of the cartridge, for example as part of a recycling process, is made more difficult due to contamination with foreign material.

### SUMMARY OF THE INVENTION

A primary object of the invention was to specify an improved vaporiser system for vaporising a composition which eliminates or at least diminishes at least one of the disadvantages of the prior art described above.

The desired improvement can relate in particular to one or more, preferably all, of the problems listed below: (i) to prevent at least one disadvantage associated with using wick/coil systems and to specify a more controllable vaporiser system; (ii) to specify a vaporiser system which generates as little waste as possible when used and is recyclable, at least in large parts; (iii) to specify a vaporiser system which is particularly easy to make ready for operation again after use and which at the same time has a particularly high degree of operational reliability that minimises risks to the health of the user and maximises the operational reliability and durability of the device; (iv) to enable safe storage and easy transport of the vaporiser system; (v) to specify a vaporiser system which does not require any complex and cost-intensive electrical contacting and which does not show any signs of wear even after repeated use; (vi) to specify a vaporiser system which permits a high degree of flexibility in the configuration of the elements and the cartridge used for vaporising, as well as with regard to their arrangement in a vaporiser system; (vii) to specify a vaporiser system which has a high degree of leak tightness against unintentional leakage of the composition and allows safe operation even without the use of expensive seals; (viii) to specify a vaporiser system in which comparatively fewer cost-intensive components have to be arranged in the cartridge, preferably achieving high recoverability and/or recyclability of the cartridge. At the very least, however, the object was to specify an alternative vaporiser system.

A secondary object of the invention was to specify a cartridge for a corresponding vaporiser system, a portable vaporising apparatus comprising a corresponding vaporiser system, an absorber for a corresponding vaporiser system, a composition for a corresponding vaporiser system, a spatial juxtaposition of a plurality of components of a corresponding vaporiser system, as well as a method for vaporising a composition in a vaporiser system and uses of corresponding cartridges, absorbers and compositions in corresponding vaporiser systems.

At least one of each of the objects referred to above is achieved by a vaporiser system for vaporising a composition, cartridges, a portable vaporising apparatus, an absorber, a composition, the spatial juxtaposition of a plurality of components of a corresponding vaporiser system, a method and uses as defined in the independent claims. Preferred embodiments according to the invention emerge from the dependent claims.

Such features of objects, compositions, methods and uses according to the invention, which are subsequently referred to as preferred, are combined in particularly preferred embodiments with other features referred to as preferred. Thus combinations of two or more of the objects, compositions, methods and uses subsequently referred to as particularly preferred are most preferred. Features referred to subsequently as preferred for vaporiser systems according to the invention are also preferred features of corresponding cartridges, vaporising apparatuses, compositions, methods and uses or the juxtaposition of a plurality of components of a corresponding vaporiser system.

The inventors of the present invention have recognised that the objects presently described can be achieved if a vaporiser system is completely separated into a primary energy “generating” part and an energy-converting secondary energy “generating” part. In this case, the primary energy is provided in the form of electromagnetic radiation which is emitted by a radiation source and is converted by an absorber arranged in the cartridge into secondary energy, which may be thermal energy and/or electromagnetic radiation with increased wavelength compared to the absorbed electromagnetic radiation, which then causes vaporising of the composition.

The invention is thus based on the concept of supplying energy to the composition in the cartridge not in the form of direct thermal energy which has been converted by a heating element as primary energy from electrical energy, but rather via electromagnetic radiation which has been emitted by a radiation source in the reusable part by means of electrical energy and is only converted into thermal energy (or electromagnetic radiation with increased wavelength compared to the absorbed electromagnetic radiation) in an absorber in the cartridge. This construction is fundamentally different from prior art vaporiser systems in which an electrical heating element in the cartridge is mechanically and electrically connected to an electrical energy source in the reusable part in order to convert electrical energy directly into the thermal energy required for vaporising.

According to a first aspect, the invention relates to a vaporiser system for vaporising a composition, comprising:

a first element comprising at least one radiation source connected to an electrical energy source, which radiation source is adapted to emit electromagnetic radiation, and

a second element comprising at least one reservoir for holding the composition and at least one absorber which is adapted to at least partially absorb the electromagnetic radiation emitted by the radiation source and to convert it at least partially into thermal energy and/or to emit it at least partially as electromagnetic radiation with increased wavelength compared to the absorbed electromagnetic radiation,

the first and the second element being reversibly and detachably connectable to each other in a non-destructive manner and a radiation conductor being arranged such that a radiation-conducting connection is formed between the radiation source and the absorber when the first element and the second element are connected to each other,

the vaporiser system being adapted to vaporise the composition by means of the thermal energy obtained from the absorber due to conversion from the electromagnetic radiation and/or by means of the electromagnetic radiation with increased wavelength compared to the absorbed electromagnetic radiation which is emitted by the absorber.

Vaporiser systems according to the invention are suitable and intended for vaporising a composition, which composition may be solid or liquid. Accordingly, in the context of the present invention, the term vaporising also includes sublimating, i.e. the direct conversion of a solid to the gaseous phase by supplying thermal energy.

The vaporiser system according to the invention comprises a first and a second element which represent structurally separate elements. The first element which is typically a reusable part, i.e. a part which is used more than just once by the subsequent customer, comprises a radiation source electrically connected to an electrical energy source, the first element preferably also comprising the electrical energy source. However, it is also conceivable that the electrical energy source is arranged in a further, separate element that can be detachably connected to the first or second element in a non-destructive manner. The radiation source is adapted to emit electromagnetic radiation.

The second element which is typically a disposable part, i.e. an element which is only used once by the consumer and is disposed of after use, is also referred to by the person skilled in the art as a cartridge. The second element, or cartridge, comprises at least one reservoir for holding the composition; in a preferred embodiment, the second element also contains the composition in the reservoir.

In addition, the second element comprises at least one absorber, the term absorber describing the absorption properties of the material with respect to the absorption of electromagnetic radiation and does not require that the absorber be capable of other types of absorption, for example the absorption of liquid.

The absorber is adapted to at least partially absorb the electromagnetic radiation emitted by the radiation source arranged in the first element. The concept of the absorption of electromagnetic radiation in condensed matter is known to the person skilled in the art. According to the invention, the absorber is adapted such that it converts the electromagnetic radiation at least partially into thermal energy. A typical everyday example to illustrate this process is a black surface which, when exposed to sunlight, heats up due to absorption of the incident radiation as a result of the thermal energy generated by conversion. This principle is also known to the person skilled in the art. Moreover, the absorber is adapted to additionally or alternatively emit the absorbed radiation at least partially as electromagnetic radiation with increased wavelength compared to the absorbed electromagnetic radiation. A corresponding wavelength shift is sometimes also referred to as a Stokes shift and is known to the person skilled in the art as an effect that can occur, for example, in fluorescence or phosphorescence. Accordingly, vaporiser systems according to the invention which comprise a fluorescent or phosphorescent absorber are preferred.

Matter absorbs electromagnetic radiation to different degrees at different wavelengths. At the same time, each matter can have a plurality of different absorption maxima along the entire electromagnetic spectrum. An absorption maximum exists when the first derivative of the absorption curve with respect to the wavelength is zero and the second derivative is not equal to zero. The highest absorption maximum is the absorption maximum at which the absorption reaches its maximum value, preferably based on a wavelength interval of 1 cm to 120 nm, particularly preferably between 1 mm and 200 nm, most preferably between 50 μm and 280 nm. Since many radiation sources are also not monochromatic and accordingly emit a spectrum, the above statements apply accordingly to emission maxima and the wavelength of the highest emission of the radiation source.

Within the scope of the present invention, the expression “at least partially” means at least 10%, preferably at least 30%, particularly preferably at least 50%, most preferably at least 70%.

In other words, the foregoing statements mean that the absorber is adapted to heat up due to the thermal energy which is obtained by conversion of the electromagnetic radiation emitted by the radiation source and absorbed by the absorber and/or to heat the surrounding composition due to the emitted electromagnetic radiation with increased wavelength compared to the absorbed electromagnetic radiation, or that the absorber is adapted to generate the emitted electromagnetic radiation with increased wavelength compared to the absorbed electromagnetic radiation by a Stokes shift from the electromagnetic radiation of the radiation source.

Preferred are vaporiser systems comprising exactly one radiation source and exactly one absorber and exactly one reservoir, as these vaporiser systems are particularly inexpensive to manufacture and particularly simple in their construction.

The first and the second element are configured such that they can be reversibly and detachably connected to each other in a non-destructive manner. The first and/or the second element preferably comprises fastening means suitable for this purpose, in particular hooks and eyes, click connections, plug-in connections, clamp connections, bayonet connections or screw connections or any combination thereof.

Within the scope of the present invention, two elements which cannot be reversibly and non-destructively detached from and reconnected to each other by the user applying usual forces, i.e. forces that can be applied with the hands, if necessary using a tool, such as a screwdriver for example, are not considered to be reversibly and detachably connectable to each other in a non-destructive manner. Within the scope of the present invention, the expression reversibly and detachably in a non-destructive manner relates to the component used for connection or fastening, for example the screw thread. It is not impossible for intentional changes to occur in the first and/or second element when connecting the first and second element but these changes do not affect the connectability and detachability. For example, it may be necessary to remove a protective film from the second element before connecting it. In some preferred embodiments, the first element, for example, comprises a spike or similar structure which is used to intentionally puncture a protective film attached to the second member or a different pierceable component during the connection process. However, it is clearly preferable for no structural changes to occur to the first element when the first and second element are connected.

According to the invention, a radiation conductor is arranged in the vaporiser system such that a radiation-conducting connection is formed between the radiation source and the absorber when the first element and the second element are used together. This means that in the connected state, radiation can pass from the radiation source in the first element to the absorber in the second element. In other words, a radiation conductor is arranged such that a radiation-conducting connection can be formed between the radiation source and the absorber when the first element and the second element are connected to each other. Accordingly, also particularly preferred is a vaporiser system according to the invention for vaporising a composition, comprising:

a first element comprising at least one radiation source connected to an electrical energy source, which radiation source is adapted to emit electromagnetic radiation, and

a second element comprising at least one reservoir for holding the composition and at least one absorber which is adapted to at least partially absorb the electromagnetic radiation emitted by the radiation source and to convert it at least partially into thermal energy and/or to emit it at least partially as electromagnetic radiation with increased wavelength compared to the absorbed electromagnetic radiation,

the first and the second element being reversibly and detachably connectable to each other in a non-destructive manner and a radiation conductor being arranged such that a radiation-conducting connection is formed between the radiation source and the absorber,

the vaporiser system being adapted to vaporise the composition by means of the thermal energy obtained from the absorber due to conversion from the electromagnetic radiation and/or by means of the electromagnetic radiation with increased wavelength compared to the absorbed electromagnetic radiation which is emitted by the absorber.

In particular, the radiation conductor can be formed by the protective disc or lens of a light-emitting diode, or by the transparent wall of the reservoir. The radiation conductor can be configured integrally or have a plurality of components. The radiation conductor can have any appropriate design that is suitable for conducting the electromagnetic radiation emitted by the radiation source to the absorber.

Preferably, both the first and the second element each comprise a radiation conductor, the two radiation conductors being arranged so that, when the first element and the second element are connected to each other, the radiation conductors are joined to each other such that a radiation-conducting connection is formed between the radiation source and the absorber.

The vaporiser system according to the invention is adapted to vaporise the composition by means of the thermal energy obtained from the absorber due to conversion from the electromagnetic radiation and/or by means of the electromagnetic radiation with increased wavelength compared to the absorbed electromagnetic radiation which is emitted by the absorber. This means that the energy necessary for vaporising the composition occurs due to the thermal energy released by the absorber to the composition and/or occurs due to the absorption of electromagnetic radiation with increased wavelength compared to the absorbed electromagnetic radiation in the composition.

In operation, the radiation source is thus supplied with electrical energy by the electrical energy source and emits electromagnetic radiation with a spectrum determined by the design and operating principle of the radiation source. The electromagnetic radiation is conducted via the radiation conductor to the absorber which absorbs at least part of the electromagnetic radiation, it being possible for losses to occur due to reflection or scattering, for example. The absorber now converts at least part of the absorbed light into thermal energy, also referred to as heat energy, or emits electromagnetic radiation shifted to longer wavelengths, also referred to as “red-shifted”, which can be absorbed by the composition and converted therein into thermal energy. As a result of the energy input, the composition vaporises and is usually delivered to the user through a vent or channel, for example, by applying negative pressure, i.e. sucking.

Vaporising of the composition thus takes place causally due to the interaction of the irradiated electromagnetic radiation with the absorber and thus effectively indirectly. Thus, vaporising of the composition does not occur, or almost does not occur, due to the direct interaction of the electromagnetic radiation emitted by the radiation source with the composition. In particularly preferred embodiments of the vaporiser system according to the invention, the composition displays almost no absorption, i.e. less than 5%, preferably less than 1%, more preferably less than 0.5%, most preferably less than 0.1%, of the maximum absorption at the wavelength of the highest emission of the radiation source. In these cases, the electromagnetic radiation of the radiation source displays almost no interaction with the composition and reaches the absorber directly even if part of the composition is in the radiation path. To facilitate better understanding, this means that the vaporiser system according to the invention, in the absence of sufficient energy transfer between the radiation source and the composition, is not suitable for vaporising a composition if the absorber is removed or if the absorber does not display absorption at a wavelength that is emitted by the selected radiation source.

Since in vaporiser systems according to the invention, an appreciable quantity of energy is imparted to the second element via the electromagnetic radiation, it is particularly preferable if the further components in the second element, i.e. the radiation-conducting components other than the absorber, display no or only very low absorption, preferably of less than 5%, particularly preferably less than 1%, particularly preferably less than 0.5% of the maximum absorption, at the wavelength of the irradiated electromagnetic radiation.

So far, own experiments have shown that, in many cases, conversion of the absorbed radiation into thermal energy in the absorber is probably the major contributor to vaporisation of the composition. Accordingly, vaporiser systems according to the invention are preferred, the absorber being adapted to at least partially absorb the electromagnetic radiation emitted by the radiation source and to convert it at least partially into thermal energy, the vaporiser system being adapted to vaporise the composition by means of the thermal energy obtained from the absorber due to conversion from the electromagnetic radiation.

Nevertheless, it can be assumed that the absorber will always also emit at least a small part of the absorbed electromagnetic radiation as wavelength-shifted radiation. The inventors have realised that this can be exploited for particularly efficient vaporisation. The idea here is that if the radiation source emits electromagnetic radiation with a wavelength which is not, or nearly not, absorbed by the composition, the wavelength shift in the absorber can advantageously cause the wavelength of the radiation to be shifted into a range where the composition does show sufficient absorption. An example of this would be the use of a blue radiation source with a wavelength of approximately 450 nm, at which a typical liquid of an e-cigarette displays only very low absorption. Due to red-shifting in the absorber, the absorber emits radiation with a longer wavelength which can be absorbed by the liquid, so that energy can be fed to the composition by this, so to speak secondary, electromagnetic radiation.

Suitable materials for the absorber are selected by the person skilled in the art without constraint on the basis of his expertise, whereby for many applications in the visible light range and adjacent spectral ranges a dark colour, e.g. dark green, dark red or dark blue, or black indicates a high absorptivity in the relevant wavelength range and thus indicates a basic suitability as an absorber within the scope of the invention. Own experiments have shown that the principle of the invention can be used for a wide range of radiation sources and electromagnetic radiations, whereby the person skilled in the art can select a suitable absorber based, in case of doubt, on absorption values, or absorption spectra, tabulated in standard works. Components which are not sufficiently absorbent per se can be coloured, dyed or coated using typical colour pigments, black pigments in particular, such as carbon black, being inexpensive, readily available and suitable. Particularly preferably, the absorber accordingly comprises colour pigments, with natural dyes especially, such as chlorophyll, being particularly preferred.

The reservoir is preferably a tank. In the case of a solid composition, the reservoir can be formed by a suitable holder, e.g. a clamp, or receptacle for the solid.

After the composition contained in the reservoir has been completely or nearly completely vaporised, the user can detach the connection between the first and the second element so that only the second element has to be replaced with a new second element which in turn is filled with fresh composition. As the shelf life of most radiation sources, particularly that of the light-emitting diodes preferred within the scope of the present invention, is particularly long, the only regular maintenance task occurring for the first element is that of recharging the energy storage device.

In particular, the vaporiser system according to the invention avoids the disadvantages associated with using customary wick/coil systems since it does not need to use a coiled filament. In particular, the radiation source used can be regularly controlled with particular precision and, if necessary, it can easily be fine-tuned by using filters, lenses and similar components. Due to the configuration as a first and second element, i.e. as a reusable part and a disposable part, the waste arising during use is minimised since a large part of the vaporiser system, i.e. at least the reusable part, is recyclable. In addition, the arrangement according to the invention makes it particularly easy for the vaporiser system to be rendered operational again, after use and complete vaporisation of the composition in the second element, merely by replacing the second element. In this respect, it is advantageously possible to use prefabricated and sealed cartridges as the second element, where the user has no access to the reservoir and the composition contained therein and is also not forced to perform a refilling step for continued operation of the vaporiser system. This achieves a particularly high level of operating safety and minimises the risks to the health of the user.

From a warranty point of view, it is also particularly favourable for the manufacturer that the operational reliability and durability of the apparatus can thus also be maximised, since in particular no foreign particles can get into the vaporiser system. The configuration separated into two parts allows vaporiser systems according to the invention to be stored and transported in a particularly safe manner, as unwanted vaporisation of the composition is not possible in the separated state.

Due to the fact that the energy input into the composition takes place due to the interaction of the electromagnetic radiation emitted by the radiation source in the first element with the absorber arranged in the second element, advantageously there is no need for any complex and cost-intensive electrical contacting, as a result of which no or hardly any signs of wear occur, at least not in the components of the system that are central to vaporisation, even with heavy use and frequent replacement of the cartridge. In the simplest case, the arrangement can be fixed by means of fastening elements in such a manner that the radiation source arranged in the first element can irradiate through a transparent shell of the reservoir onto the absorber in the connected state. As a result, vaporiser systems according to the invention advantageously display a particularly high degree of flexibility in the configuration of the elements and the cartridge used for vaporising, as well as with regard to their arrangement in the entire vaporiser system, which cannot be implemented in traditional systems. Advantageously, it is thus only necessary that electromagnetic radiation can pass from the radiation source to the absorber which is particularly easy to implement without constraint in particular with transparent reservoirs. Advantageously, cartridges can therefore also be configured, if required, such that there is more than one fitting position, i.e. arrangement position of the cartridge with respect to the first element, when connecting to the first element, which can reduce the number of user errors during connection.

Since there is no need for any electrically conductive connection between a heating element in direct contact with the composition and the energy storage device, the vaporiser system according to the invention also displays a particularly high degree of leak tightness and enables safe operation even without the use of expensive seals. Advantageously, in vaporiser systems according to the invention, the cost-intensive components, in particular the radiation source and the electrical energy storage device, are arranged in the recyclable part. Instead of a complex heating element, the cartridge, i.e. the second element, need only have an absorber, the material of which, moreover, can be selected so as to provide a high degree of compatibility with and/or easy separability from the material of the reservoir, for example, when a coloured silicate glass is used in a reservoir of silicate glass. This improves the recyclability of the cartridge in a particularly advantageous manner.

Most preferably, and accordingly highlighted here, are vaporiser systems according to the invention, in which the emitted electromagnetic radiation has the highest intensity maximum below a wavelength of 500 nm, preferably ranging from 410 to 490 nm, preferably 430 to 480 nm, particularly preferably 440 to 470 nm, the electromagnetic radiation particularly preferably having a spectral bandwidth at 50% of the maximum intensity of 5 to 50 nm, preferably 10 to 40 nm, particularly preferably 20 to 30 nm.

The intensity of non-ideal monochromatic electromagnetic radiation is a function of the wavelength. The expression spectral bandwidth at 50% of the maximum intensity refers to the difference in wavelength between the two wavelengths to the left and right of the intensity maximum in the spectrum at which the intensity has dropped to 50% of the maximum value.

These vaporiser systems according to the invention are preferred in such a manner because they make the most advantageous use of the fact that with the invention, i.e. by using an absorber and the “indirect” energy input into the composition provided thereby, it is possible to dispense with using IR radiation, i.e. electromagnetic radiation in the IR range. Corresponding vaporiser systems are also preferred because the liquids commonly used today do not usually display any significant absorption in the radiation range specified and thus the absorber can readily be irradiated even if it is arranged in the composition. In addition, simple, black absorber materials have proven to be particularly efficient absorbers at these wavelengths. It was found to be a particularly great advantage that the specified characteristics of the electromagnetic radiation can be implemented particularly easily with particularly inexpensive and at the same time long-lasting radiation sources.

These preferred vaporiser systems thus allow the location of energy generation to be specifically adjusted and also make it possible to provide the vaporisation location on the side of the composition, or reservoir, directed away from the radiation source. In addition, it is advantageously possible to operate the preferred vaporiser systems without a radiation source for infrared electromagnetic radiation and to operate them instead with particularly inexpensive radiation sources.

The features disclosed as preferred for vaporiser systems according to the invention accordingly also apply to the vaporiser system disclosed above.

In light of these statements, it is apparent to the person skilled in the art that vaporiser systems according to the invention are preferred, the emitted electromagnetic radiation having the highest intensity maximum below a wavelength of 500 nm, and the composition displaying almost no absorption, i.e. less than 5%, preferably less than 1%, more preferably less than 0.5%, most preferably less than 0.1% of the maximum absorption, at the wavelength of the highest intensity maximum and/or the absorber displaying an absorption of more than 50%, preferably more than 75%, particularly preferably more than 95% of the maximum absorption at the wavelength of the highest intensity maximum, vaporiser systems according to the invention with the AND relation of the energy conversion type being particularly preferred.

In light of the prior art discussed above, it is self-evident to the person skilled in the art that this knowledge can also be implemented advantageously in vaporiser systems that are configured in only one part. Accordingly, a vaporiser system for vaporising a composition is disclosed, comprising:

at least one radiation source connected to an electrical energy source, which radiation source is adapted to emit electromagnetic radiation,

at least one reservoir for holding the composition, and

at least one absorber which is adapted to at least partially absorb the electromagnetic radiation emitted by the radiation source and to convert it at least partially into thermal energy and/or to emit it at least partially as electromagnetic radiation with increased wavelength compared to the absorbed electromagnetic radiation,

a radiation conductor being arranged such that a radiation-conducting connection exists between the radiation source and the absorber,

the vaporiser system being adapted to vaporise the composition by means of the thermal energy obtained from the absorber due to conversion from the electromagnetic radiation and/or by means of the electromagnetic radiation with increased wavelength compared to the absorbed electromagnetic radiation which is emitted by the absorber,

the emitted electromagnetic radiation having the highest intensity maximum below a wavelength of 500 nm, preferably ranging from 410 to 490 nm, preferably 430 to 480 nm, particularly preferably 440 to 470 nm, the electromagnetic radiation particularly preferably having a spectral bandwidth at 50% of the maximum intensity of 5 to 50 nm, preferably 10 to 40 nm, particularly preferably 20 to 30 nm, and

the composition preferably displaying almost no absorption, i.e. less than 5%, preferably less than 1%, more preferably less than 0.5%, most preferably less than 0.1% of the maximum absorption, at the wavelength of the highest intensity maximum and/or the absorber preferably displaying an absorption of more than 50%, preferably more than 75%, particularly preferably more than 95% of the maximum absorption at the wavelength of the highest intensity maximum.

A vaporiser system according to the invention is preferred, the absorber being a three-dimensional body, the dimension of which in two spatial directions is greater than or at least equal to the dimension in the third spatial direction, preferably a plate with any base area, in particular a disc, or a cuboid, the absorber preferably having at least one planar or curved surface, preferably at least two, particularly preferably at least four planar surfaces,

or

the vaporiser system comprising a composition and the absorber being formed by particles which are mixed with the composition to be vaporised or dispersed in the composition to be vaporised.

Corresponding vaporiser systems according to the invention have proven to be particularly advantageous in own tests. Advantageously, the absorber has at least one planar or curved surface which the radiation source can irradiate particularly efficiently in order to thus ensure the greatest possible interaction between the electromagnetic radiation and the absorber. Clearly preferred in this respect are flat structures with at least one planar or substantially planar surface, through which losses due to scattering and reflection can be prevented in the best way possible.

Corresponding vaporiser systems according to the invention with a solid, macroscopic absorber are particularly easy to control and allow particularly fine adjustment of the radiation path and energy transfer. Corresponding absorbers are preferred because they regularly have a high ratio of irradiated surface to absorber mass. Alternatively, the absorber can be provided directly with the composition to be vaporised. In this case, it is sometimes more difficult to irradiate the absorber particles in a targeted manner, but as the absorber is not fixedly connected to the cartridge, the absorber can subsequently be removed from the cartridge without leaving any residue, thus preserving particularly favourable recycling properties.

A vaporiser system according to the invention is preferred, the absorber being configured such that one or more of its absorption maxima for electromagnetic radiation lie at a wavelength of the electromagnetic radiation which is emitted by the radiation source, preferably at a wavelength that lies within 20%, preferably within 10%, particularly preferably within 5%, around an intensity maximum of the emission of the radiation source.

Corresponding vaporiser systems are preferred because particularly high efficiency is achieved when the absorber is matched quite precisely to the electromagnetic radiation emitted by the radiation source. In corresponding vaporiser systems, energy losses can be minimised and the radiation energy used is introduced into the composition particularly efficiently via the absorber, such that particularly long operating times can be achieved.

A vaporiser system according to the invention is preferred, the absorber having channels, preferably capillary channels, and/or being a porous solid body, preferably having capillary channels, such that the absorber is liquid-conducting and passage of the liquid composition, or of the gaseous composition, through the absorber is possible, the absorber preferably comprising a membrane which only allows passage of the liquid composition into the absorber or through the absorber when a limit temperature is exceeded. Preferred examples of such absorbers are sintered, open-pored glass, sintered open-pored ceramics, structured components with channels made by processes of the semiconductor industry, open-pored foams, loose granular grains in bulk, held in a suitable liquid-permeable section.

A corresponding absorber is most particularly advantageous because it has a large surface area which can be wetted by the composition so that the thermal energy can be transferred particularly efficiently to the composition. Correspondingly configured absorbers can additionally be used as a separating wall between the reservoir for storing the composition and the vent, i.e. the channel for the vaporised composition, it being possible due to the preferred configuration to obtain second elements which are particularly leak-tight outside of use in the vaporiser system and are secured against undesirable leakage of the composition. With the preferred absorbers, the composition guided to the absorber can pass through it and is vaporised when the radiation source is activated, the resulting vapour being able to pass through the channels into the vent and to the outlet opening.

A vaporiser system according to the invention is preferred, the absorber having a non-homogeneous absorption behaviour along at least one spatial direction, preferably a gradient of absorption along the spatial direction corresponding to the direction of incidence of the electromagnetic radiation onto the absorber, the gradient of absorption preferably being generated by a concentration gradient of pigments with an absorption maximum at the wavelength of the electromagnetic radiation in an absorber which is otherwise or largely transparent at this wavelength.

Corresponding vaporiser systems according to the invention are preferred because they enable a particularly high degree of freedom in the configuration and arrangement of the components. A corresponding absorber can also be exposed to electromagnetic radiation from the side and still absorb it over a large area. Although the radiation intensity decreases when the electromagnetic radiation passes through the absorber, the absorptivity increases so that, depending on the course of the absorption gradient, it is easy to adjust the desired absorption profile and thus the profile of the thermal energy in the absorber. Alternatively, a corresponding absorption gradient in the absorber also offers the possibility of adjusting a desired temperature profile, i.e. a profile of the thermal energy emitted, on the absorber in a targeted, spatially resolved manner, i.e. deliberately providing warmer (higher absorption) and colder (lower absorption) regions during irradiation. Advantageously, this can also be optimised by selecting a suitable thermal conductivity of the absorber, e.g. a lower thermal conductivity compared to the composition. Corresponding absorbers can be produced by a materials scientist without great effort and can be generated, for example, using carbon black particles or suitable doping in a glass or crystal matrix. The expression “largely transparent” in the context of the present invention means that, at the corresponding wavelength, there is an absorption of less than 5%, preferably less than 2%, more preferably less than 1%, particularly preferably less than 0.5% of the maximum absorption.

A vaporiser system according to the invention is preferred, the radiation source being a lamp, a laser or a light-emitting diode, preferably a laser or a light-emitting diode, particularly preferably a light-emitting diode, the laser preferably being a laser diode, a fibre laser or a gas laser and the light-emitting diode preferably being a semiconductor light-emitting diode (LED), an organic light-emitting diode (OLED) or a chip-on-board light-emitting diode (COB-LED).

Corresponding vaporiser systems are preferred because the specified radiation sources have proven to be particularly efficient in practice at implementing the invention. The use of a light-emitting diode has proved to be most preferable, as it is not only particularly long-lasting and energy-saving but also requires comparatively little expenditure on equipment. It was quite surprising from the point of view of the inventors that using a light-emitting diode is sufficient within the scope of the invention to enable meaningful vaporisation, and that a monochromatic and high-energy laser is not necessarily required.

Alternatively, a vaporiser system according to the invention is preferred, the electromagnetic radiation in the radiation source being generated by induction.

A vaporiser system according to the invention is preferred, the radiation conductor being opaque to electromagnetic radiation with a wavelength which deviates more than 50%, preferably more than 30%, particularly preferably more than 10% from the wavelength of the intensity maximum of the electromagnetic radiation emitted by the radiation source.

In the context of the present invention, the expression opaque means that a material is not transparent, or is substantially not transparent. Accordingly, opaque means that the absorption at a given wavelength is more than 90%, preferably more than 98%, more preferably more than 99%, particularly preferably more than 99.5%. Corresponding vaporiser systems are preferred because they are particularly secure against unwanted energy input into the composition. Corresponding vaporiser systems can be configured so that substantially only the intended electromagnetic radiation provided by the radiation source reaches the absorber and not, for example, any stray light from the environment. This allows particularly reliable control of the energy supply to the composition and advantageously increases the storage stability.

A vaporiser system according to the invention is preferred, the absorber having at least one planar surface, preferably two planar surfaces, particularly preferably six planar surfaces, and wherein the radiation source, the radiation conductor, any radiation formers that may be present and the absorber, when the first and second elements are connected to each other, are arranged in such a way that the electromagnetic radiation impinges on one of the planar surfaces of the absorber at an angle of incidence of less than 45°, preferably less than 20°, particularly preferably less than 5°, most preferably substantially perpendicularly.

Own studies have shown that the appropriate relative arrangement of the elements to each other is advantageous because radiation or energy losses due to unwanted reflection or scattering are advantageously minimised in these systems and absorption in the absorber is often particularly uniform.

A vaporiser system according to the invention is preferred, the reservoir being transparent at least in one section, preferably transparent to visible light, particularly preferably transparent to electromagnetic radiation, the wavelength of which is within 20%, preferably within 10%, particularly preferably within 5%, around the intensity maximum of the emission of the radiation source.

Corresponding vaporiser systems according to the invention are advantageous because they not only allow the user to check the level in the reservoir from the outside, but they also particularly advantageously allow the electromagnetic radiation emitted by the radiation source to pass directly through the wall of the reservoir so that the absorber can be arranged behind the reservoir in or with respect to the radiation source, which particularly increases flexibility in the arrangement of the elements.

A vaporiser system according to the invention is preferred, comprising a first absorber and a second absorber as well as a first radiation source and a second radiation source, wherein the first and the second absorber are preferably connected to different, separate sections of the reservoir and wherein the first and the second radiation source have their highest emission maximum preferably at different wavelengths, wherein the absorptivity of the two absorbers preferably differs at at least one of the wavelengths of the highest emission maximum of the two radiation sources by more than 50%, preferably by more than 70%, particularly preferably by more than 85%.

The use of two or more absorbers, preferably made of different absorber materials, is already preferred per se, since this makes it possible to influence either the intensity of the vaporisation by controlled guidance of the electromagnetic radiation, namely by how many absorbers are irradiated, or it also makes it possible to selectively control different absorbers that are in contact with different reservoirs or spatially separated sections of the same reservoir. For example, it is thus possible to selectively initiate a first vaporisation in a first absorber in order to already initiate a second vaporisation in a second absorber during the cooling phase of the first absorber, so that the vaporiser system has a very low latency and can provide quasi-continuously vaporised composition portions in a precisely adjusted concentration.

Most particularly advantageously, it is combined with two radiation sources so that the intensity of the vaporisation can be controlled by switching the additional radiation source on and off. It is also most particularly favourable if the two radiation sources have different emission characteristics, i.e. if one or more emission maxima are at different wavelengths, since this enables different operating modes (provided that the absorber has different absorptivities for these wavelengths). If the two absorbers have a different absorptivity at at least one of the highest emission maxima of the two radiation sources, preferably at both wavelengths of the highest emission maxima, then the resulting vaporiser system can be controlled particularly efficiently in this way. By selectively activating one or both radiation sources, the two absorbers can in fact be addressed simultaneously, if necessary with different intensity, or individually, and thus it can be determined, for example, from which reservoir vaporisation is to take place.

A vaporiser system according to the invention is preferred, the vaporiser system being suitable for use in a portable vaporising apparatus, preferably a hand-held device, preferably in an e-cigarette or an inhaler, e.g. for medical purposes, the first element preferably being configured as a reusable part and the second element preferably being configured as a disposable part, the second element preferably being a cartridge. Use for medical purposes includes in particular the application of drugs for respiratory diseases as well as painkillers. Preferably, the two different emission maxima of the radiation sources which are matched to the two absorbers are implemented in the form of a single two-colour light-emitting diode. This achieves a big reduction in the installation space required for the radiation source, while at the same time utilising the advantages described above.

While in known inhalers for powdered drugs, the inhaler usually only needs to be cleaned before and/or after use, or even maintained beyond that, this is usually not necessary in a vaporiser system according to the invention, particularly as the drug, whether in solid or liquid form, is enclosed in the reservoir.

In addition, a vaporiser system according to the invention can be used for various therapies, for which purpose either the contents of the reservoir are introduced or replaced accordingly with regard to the therapy to be applied, or expediently an empty component corresponding to a second element according to the invention or one filled with a first drug is exchanged for another component likewise corresponding to a second element according to the invention.

Individual, in particular manual, loading and preceding or subsequent cleaning of the inhaler for each dose or application can also be omitted if the reservoir is configured to be large enough to accommodate a sufficient quantity of the composition for several doses or applications.

Since in the vaporiser or inhaler according to the invention, the drug in the form of vapour is usually released completely into the (respiratory) air flow of the user used for inhalation, a number of further advantages can be implemented: In particular, there are typically no residues of the drug remaining in the inhaler after use because the drug vapour only, at least substantially, condenses in the air flow. In addition, particularly good application success can be achieved, as the function according to the invention allows the maximum quantity of drug that can be absorbed by the patient to be dispensed into the inhalable air flow. It can also prevent overdosing of the drug during subsequent use of the inhaler, due to taking any drugs not removed, which is particularly advantageous in terms of increased safety for the patient. It also allows for an increase in the control options when treating the patient as more precise dosing is possible.

In addition, the vaporiser system according to the invention does not require dispersants or propellants (in particular propellant gases) which can often be unfavourable or even hazardous to health, especially in a medical environment. In this context, a propellant gas is understood in particular as a gas which has an increased pressure compared to the ambient pressure and which is used in typical customary inhalers to atomize and accelerate the medication to be applied. It is also possible to dispense with a compression device for generating an air flow or other gas flow and with the associated dead volumes in the housing of the inhaler. In this way, a reduction in the required installation space can be achieved as only one air channel is needed instead of space-occupying pressure chambers or complex spring systems for pre-charging the inhaler. The lower installation space requirements can in turn be advantageous, particularly with regard to an increase in configuration freedom thus made possible in the design of the inhaler (e.g. smaller, more appealing designs are possible).

In a vaporiser system or inhaler according to the invention, on the other hand, the user can transfer the drug to the lungs using their own inhaled air after the drug has been vaporised. Due to the fact that there is no need to use propellants to atomize the medication, problems frequently encountered by patients when using customary inhalers with propellants, such as irritation of the throat or coughing, can be effectively prevented. It also eliminates the need to manually pre-charge the inhaler, which is often required with customary inhalers, in order to atomize the drug. In this way, user friendliness can be improved, in particular by avoiding manual preparatory actions before using the inhaler.

Since the vaporiser system according to the invention, in particular its radiation source, is electrically operated, a high dosing accuracy can be achieved due to the precise controllability of the activity duration of the radiation source. This particularly promotes the application quality, as the vaporised quantity of the drug can be very easily adapted to the individual needs of the user or patient and, in particular, can also be limited in terms of a maximum dose, which in turn can be used to increase the application safety when using the vaporiser system or inhaler.

A vaporiser system according to the invention is preferred, the reservoir comprising one or more materials which are selected from the group consisting of glass, crystal, metal, ceramic, wood and plastic, the reservoir preferably having a further outer shell.

A vaporiser system according to the invention is preferred, the reservoir being formed by a bag, the bag being made entirely or partially of silicone, rubber, latex or other suitable elastic or non-elastic material, preferably a plastic. The use of bags as reservoirs is particularly advantageous, as they are inexpensive to produce and regularly generate only small quantities of waste. In addition, it is advantageously not necessary to provide pressure equalisation in the reservoir, as the bag will contract if necessary while the internal pressure remains constant. In addition, bags are advantageous for certain applications because they do not splinter and are therefore associated with less potential risk.

A vaporiser system according to the invention is preferred, the preferably rigid reservoir being equipped with an element for pressure equalisation.

A vaporiser system according to the invention is preferred, the electrical energy source being an energy storage device, preferably a battery or a fuel cell, particularly preferably a lithium-ion battery, in particular a rechargeable lithium polymer battery.

A vaporiser system according to the invention is preferred, the radiation source being adapted to emit electromagnetic radiation with an intensity that is suitable so that the portion of the emitted electromagnetic radiation absorbed and converted by the absorber can evaporate at least 3 to 9 mg, preferably 5 to 7 mg of the composition, preferably exactly a predetermined quantity of the composition, in 1 to 5 s, preferably in 2 to 4 s, particularly preferably in 2.5 to 3.5 s.

Corresponding vaporiser systems are preferred because in comprehensive tests with consumers who were asked to evaluate the vapour experience with a liquid containing nicotine, it was shown that the specified quantities of vaporised composition are mostly perceived as advantageous for the vapour sensation, particularly when compared to smoking a cigarette. Conveniently, the vaporised quantity of composition in the vaporiser system according to the invention can be adjusted very accurately and reliably by adjusting the power of the radiation source.

A vaporiser system according to the invention is preferred, the radiation source being adapted to be operated continuously or pulsed, pulsed operation being preferred, preferably with pulse durations ranging from 0.2 ms to 2000 ms, preferably 1 ms to 1000 ms, more preferably 10 ms to 500 ms, most preferably 10 ms to 100 ms. For certain applications, in particular those using an LED as the radiation source, it is preferred to select the pulse duration in the range of 0.5 to 20 ms, preferably 1 to 10 ms. Preferably, the pulse durations are selected depending on the thermal time constant of the absorber. In this context, the thermal time constant describes the absorber-specific time in which the thermal energy emitted by the absorber has dropped to 50% of the previously absorbed energy. In own test series, it has been shown that pulsed operation of the radiation sources regularly ensures better energy input into the absorber and thus indirectly enables better energy input into the composition. Without wanting to be bound to this theory, this is attributed to the fact that to a certain extent the absorber has time between the pulses to strive towards the equilibrium state.

Preferably, the radiation source can be operated continuously during a duty cycle and pulsed during a time period following the duty cycle. The duty cycle preferably has a duration of 1 ms to 1000 ms, preferably 10 ms to 1000 ms, more preferably 100 ms to 1000 ms.

A vaporiser system according to the invention is preferred, the radiation source being adapted to function as a radiation sensor, in particular as an infrared sensor, in a second operating mode. Corresponding vaporiser systems are particularly advantageous because they can also be used to detect radiation without the addition of a further component and with only minimal changes in the control of the radiation source used to input energy into the absorber. This can be used, for example, to make a statement about the temperature of the components in the first element by detecting infrared radiation.

A vaporiser system according to the invention is preferred, the vaporiser system comprising at least two radiation sources which can preferably be controlled separately and independently of each other.

Vaporiser systems according to the invention with a radiation source for monochromatic electromagnetic radiation are particularly precise to adjust and allow the absorber to be tuned very accurately to the specific wavelength of the electromagnetic radiation. However, in view of the frequently complicated equipment-related requirements and the susceptibility to vibrations and contamination, such mostly laser-based vaporiser systems are currently of interest primarily for stationary vaporiser systems, in which the first element can be configured to be sturdier and can also reliably accommodate vibration-sensitive components. Even though it is conceivable in principle for mobile applications to generate quasi-monochromatic electromagnetic radiation by using filters, this can be accompanied by undesirable losses in efficiency. It is therefore particularly advantageous to basically use radiation sources that are inherently not monochromatic but have a low spectral bandwidth per se. In practice, however, it has been shown that, from a cost-benefit point of view, it is often not advisable to invest too much money in an effort to achieve even further minimal improvement in the range of already low spectral bandwidths

A vaporiser system according to the invention is preferred, the electromagnetic radiation being monochromatic or having at least 90% of the intensity in a wavelength range of ±20%, preferably ±10%, particularly preferably ±5%, around the intensity maximum, most preferably has a spectral bandwidth at 50% of the maximum intensity of 5 to 70 nm, preferably 10 to 50 nm, particularly preferably 15 to 30 nm, around the intensity maximum.

A vaporiser system according to the invention is preferred, the electromagnetic radiation the highest intensity maximum in the wavelength range between 10 cm and 120 nm, preferably between 1 cm and 200 nm, particularly preferably between 1 mm and 280 nm, most preferably between 50 μm and 380 nm, particularly preferably between 500 nm and 350 nm.

As explained above, the invention is also so advantageous because the underlying concept can be used in principle for a wide range of electromagnetic radiations. However, from the point of view of everyday practicability, especially taking safety aspects into account, it is clear why using radiations in the range indicated above, in particular between infrared and UV, is particularly preferred.

A vaporiser system according to the invention is preferred, the absorber being adapted to absorb at least 50%, preferably at least 75%, particularly preferably at least 90%, of the electromagnetic radiation emitted by the radiation source and to convert at least 20%, preferably at least 50%, particularly preferably at least 75%, most preferably at least 90%, into thermal energy and/or to emit at least 20%, preferably at least 50%, particularly preferably at least 75%, most preferably at least 90%, as electromagnetic radiation with increased wavelength compared to the absorbed electromagnetic radiation, providing that never more than 100% of the absorbed electromagnetic radiation is converted.

Corresponding vaporiser systems are preferred because they minimise losses due to insufficient absorption, it being possible to adapt the absorber accordingly by selecting a suitable material or suitable coating, as well as an appropriate geometry and surface structure which should be matched in particular to the arrangement to the radiation source. It is also possible due to the choice of material for the person skilled in the art to control whether the radiation goes mainly into thermal energy or into emitted electromagnetic radiation with increased wavelength.

A vaporiser system according to the invention is preferred, the absorption properties of the absorber being generated and/or modified by a coating.

Corresponding vaporiser systems are most preferred because own experiments have fortunately shown that the absorption properties of the absorber can be selectively controlled by selecting suitable coatings. In particular, by coating otherwise non-absorbent components, for example parts of the reservoir, it is possible to create an absorber, in the sense of the present invention, from them, at least in sections. Suitable coatings are matched to the radiation source by selecting appropriate dyes. Suitable pigments for a corresponding coating include, for example, Vantablack, a carbon nanotube-based material, or pigments such as Mars Black (an iron oxide pigment), carbon black, charcoal, nuclear black, slate black or Frankfurt black. Alternatively, the surface of components provided as absorbers can also be selectively increased in their absorption by a suitable surface treatment, for example by chemical etching of a nickel-phosphorous alloy, the resulting surface being known as super black.

A vaporiser system according to the invention is preferred, the absorber having a structured surface with a mean surface roughness Ra ranging from 0.2 μm to 1 mm, preferably 1 μm to 500 μm, preferably ranging from 2 μm to 100 μm, so that the wettability of the absorber and/or the absorptivity of the absorber with respect to the absorbed electromagnetic radiation is changed, in particular is improved.

A vaporiser system according to the invention is preferred, the absorber having a membrane which can be caused to mechanically vibrate due to interaction with the electromagnetic radiation and is thereby suitable for atomising the liquid composition.

A vaporiser system according to the invention is preferred, the absorber having, at least in sections, a high thermal conductivity of more than 0.3 W/(m*K), preferably more than 20 W/(m*K), further preferably more than 100 W/(m*K), the absorber preferably also having in sections a low thermal conductivity of less than 10 W/(m*K), preferably less than 5 W/(m*K), more preferably less than 0.5 W/(m*K), very preferably less than 0.3 W/(m*K). High thermal conductivity leads to preheating of the composition adjacent to the absorber. As a result, with a liquid composition, the viscosity of the composition can be advantageously influenced, preferably to a reduction in the viscosity. Low thermal conductivity of the absorber leads to optimised local energy input.

A vaporiser system according to the invention is preferred, the absorber being arranged in the reservoir, preferably on the base of the reservoir, recessed in the base, on a wall of the reservoir and/or recessed in a wall, the absorber preferably being enclosed by a region of the reservoir in a permanently positively bonded manner.

A vaporiser system according to the invention is preferred, the absorber forming part of the outer shell of the reservoir.

A vaporiser system according to the invention is preferred, the absorber having a low thermal capacity, particularly preferably a lower thermal capacity than the mean thermal capacity of the materials used in the reservoir, particularly preferably of the materials which are in contact with the absorber. Corresponding vaporiser systems according to the invention are preferred because an absorber with a low thermal capacity can respond particularly quickly to temperature changes, since the absorber has a so-called low thermal mass. Corresponding vaporiser systems are accordingly less sluggish than comparable systems and, after a completed vaporising interval, are available again more quickly in the initial state.

A vaporiser system according to the invention is preferred, the absorber being connected to an additional heat conductor which is adapted to conduct the thermal energy from the absorber to a vaporising region in which the composition can be vaporised, the heat conductor preferably comprising metal, semiconductor, glass, ceramic, plastic or heat pipes, the heat conductor preferably having a thermal conductivity of more than 0.3 W/(m*K), preferably more than 20 W/(m*K), further preferably more than 100 W/(m*K).

For structural reasons, it may be preferable to spatially separate generation of the thermal energy in the absorber from vaporisation of the composition. In this case, it is necessary to conduct the thermal energy generated in the absorber to a vaporisation region via a heat conductor, i.e. a region in which the composition is vaporised on the heat conductor using the thermal energy obtained from the absorber. Accordingly, corresponding vaporiser systems are preferred because they allow even further increased flexibility with regard to the arrangement of the components used in the vaporiser system.

A vaporiser system according to the invention is preferred, the vaporiser system comprising at least two separate absorbers, the two absorbers preferably being in contact with two sections of the reservoir that are separated from each other.

A vaporiser system according to the invention is preferred, the composition being solid or liquid, preferably liquid, the composition preferably being a solution, preferably an aqueous solution, an oil, a gel, a powder or a paste.

A vaporiser system according to the invention is preferred, the radiation conduction in the radiation conductor being based on total and/or partial reflection. In other words, the radiation conductor can comprise a material which conducts the electromagnetic radiation emitted by the radiation source, according to the principle of reflection of electromagnetic radiation, at the boundary surfaces of the radiation conductor. Such materials are known to the person skilled in the art in connection with optical wave-guiding through optical fibres. For example, the radiation conductor can comprise a glass, e.g. in the form of optical fibres, or a plastic, for example, PMMA or polycarbonate, which is suitable for the purpose of conducting radiation in relation to electromagnetic radiation. The radiation conductor can have both a purely light-conducting effect and an effect influencing the electromagnetic radiation, in which case it then also acts as a radiation former.

A vaporiser system according to the invention is preferred, the radiation conductor being transparent in at least one direction to at least part of the radiation emitted by the radiation source, preferably to radiation with the wavelength at the intensity maximum.

A vaporiser system according to the invention is preferred, the radiation conductor comprising evacuated sections.

A vaporiser system according to the invention is preferred, the radiation conductor comprising one or more radiation-conducting materials which are selected from the group consisting of solids, liquids and gases, preferably selected from the group consisting of glasses, plastics, mineral materials, organic liquids, air and aerosols, and particularly preferably selected from the group consisting of doped and non-doped quartz glasses, synthetic resin, polyethylene, polyurethane, polyethylene terephthalate, polypropylene, polycyclohexylene dimethylene terephthalate, mineral crystals, sapphires, rock crystals, diamond, ethylene glycol, glycerol and air.

A vaporiser system according to the invention is preferred, the radiation conductor comprising lenses, in particular concave and/or convex lenses, and/or fully reflecting or partially reflecting mirrors, in particular concave and/or convex mirrors, and/or optical resonators for adapting the radiation conduction, and/or being configured to be prism-like and/or segmented into differently conducting regions.

A vaporiser system according to the invention is preferred, the radiation conductor being configured, at least in sections, to be cuboid, spherical, annular, toroidal, disc-shaped, U-disc-shaped, strip-shaped, cube-shaped, strand-shaped, strand-shaped thickening, strand-shaped tapering, bent, curved, asymmetrical and/or symmetrical.

A vaporiser system according to the invention is preferred, the radiation conductor being arranged, at least in sections, in an annular or semi-circular shape around the absorber.

Corresponding vaporiser systems according to the invention are preferred because an at least sectionally annular arrangement of the radiation conductor around the absorber leads to particularly large-area irradiation of the absorber, which results in particularly efficient utilisation of the surface provided by the absorber, and thus, in relation to the mass of the absorber, brings about particularly high absorption and therefore generation of thermal energy and/or electromagnetic radiation with increased wavelength compared to the absorbed electromagnetic radiation.

A vaporiser system according to the invention is preferred, the radiation conductor comprising channels, preferably capillary channels, produced in a controlled manner or generated statistically, which lead through the radiation conductor, and/or comprising porous sections.

A vaporiser system according to the invention is preferred, the radiation conductor comprising regions with higher or lower degree of order which, at least in sections, have other physical properties than the remaining radiation conductor, and/or the radiation conductor preferably being opaque in sections.

A vaporiser system according to the invention is most preferred, additionally comprising a capillary or porous material, which is arranged in such a manner between the reservoir and the absorber that the transport of a liquid composition from the reservoir to the absorber is enabled by capillary forces, preferably at least one side of the absorber being completely covered by the capillary or porous material. The capillary or porous material can be a wick similar to that used in wick/coil systems.

Corresponding vaporiser systems according to the invention are preferred because transport of the liquid composition from the reservoir to the absorber is promoted by the capillary or porous material. The corresponding porous material soaks up the composition and provides it in the vicinity of the absorber so that it can be vaporised there. This not only prevents or at least slows the uncontrolled flow of composition out of the cartridge towards or through (or even past) the absorber, but also ensures, regardless of the orientation of the vaporiser system and the fill level in the reservoir, that there is always enough composition in the vicinity of the absorber to ensure sufficient vaporisation and thus also to prevent or reduce the occurrence of the Leidenfrost effect at the absorber.

A vaporiser system according to the invention is preferred, additionally comprising one or more sensor units, the one or more sensor units being selected from the group consisting of radiation sensors, in particular infrared sensors, temperature sensors, pressure sensors, flow rate sensors, ammeters, voltmeters, position sensors, mass flow sensors, volume flow sensors, level sensors for determining the level in the tank, optical sensors, chemical sensors, chemical analysis devices.

Corresponding vaporiser systems according to the invention are preferred because with them it is possible to obtain comprehensive information about the vaporiser system, even during operation. This enables particularly precise monitoring of the system and it is thus possible, for example, to detect imminent or already occurring faults at an early stage. Using suitable sensors, for example pressure sensors and flow rate sensors, it is also possible, particularly in medical applications, to ensure that patients have actually taken in the intended quantity of inhalant, i.e. of the vaporised composition. Temperature sensors or chemical sensors can also be used to determine whether the operating conditions of the vaporiser system may have led to the formation of undesirable, harmful substances or whether this can at least be assumed based on the temperature measured. One or more sensor units are preferred, linked to an emergency stop device which prevents continued operation in the event that certain measured values are exceeded.

A vaporiser system according to the invention is preferred, additionally comprising a supplementary tank for holding clean air, the supplementary tank preferably being a pressure vessel.

Corresponding vaporiser systems according to the invention are most preferred for medical applications. Deep inhalation of the vaporised composition, which is often desirable and necessary with a view to sufficient application of the active ingredient, can in principle expose the patient to increased risk, since of course other, potentially undesirable, constituents can also penetrate deeply into the lungs with the breath inhaled. However, in large cities and metropolitan areas, the air quality, particularly in terms of smog and particulate pollution, is so poor in some cases that it can be potentially unhealthy for a patient with respiratory disease to inhale so deeply. Likewise, a risk arises if the application takes place in a crowded hospital, for example, where it cannot be ruled out that diseases transmissible by droplet infection, for example, are picked up when the active ingredient is inhaled, for example from the person sitting next to him in the waiting area who is coughing. It is therefore particularly positive that the preferred vaporiser system can provide controlled clean fresh air from a supplementary tank, thereby circumventing these problems. Against the same background, it is also preferred as a supplementary or alternative embodiment, that the vaporiser system according to the invention has a supply air inlet at which an air filter is arranged which is adapted, for example, to filter out pollen or particulates. Accordingly, a vaporiser system according to the invention is preferred, additionally comprising an air filter, the air filter preferably being arranged at the supply air inlet.

A vaporiser system according to the invention is preferred, additionally comprising an outlet opening and a vent connected to this outlet opening, the vent being arranged so that the vaporised composition can pass from the absorber to the outlet opening, the vent preferably being connected to a supply air inlet through which air can pass through the vent to the outlet opening so that it can serve as a carrier medium for the vaporised composition, the vent preferably being preferably coaxially surrounded by the reservoir and preferably being formed by the walls of the reservoir, or the vent being formed at least in sections by the absorber or the vent being formed between the first and the second element.

A vaporiser system according to the invention is preferred, additionally comprising a printed circuit board and a control device for the radiation source.

A vaporiser system according to the invention is preferred, additionally comprising a radiation former which is suitable for deflecting, reflecting, scattering or focusing the electromagnetic radiation. Examples of a radiation former are optical filters, lenses, mirrors and can be found below in connection with the configuration of the radiation conductor. Corresponding vaporiser systems are preferred because the use of radiation formers for deflecting, reflecting, scattering or focusing the electromagnetic radiation considerably increases the flexibility when arranging the first and second elements with respect to each other and when arranging the components in the respective elements.

A vaporiser system according to the invention is preferred, the absorber being arranged in the vaporiser system in such a manner that the composition held in the reservoir is in contact with or can come into contact with the absorber.

A vaporiser system according to the invention can preferably comprise an output quantity control device. In particular, this can be a counting device, in particular a counting device for counting doses of the composition vaporised in a defined observation period (for example in the period since the counting device was last initialised or reset). In particular, the doses to be counted here can be (i) a number of vaporised fills of the reservoir or of different reservoirs, or (ii) a number of vapour shots or predetermined vapour quantity units emitted by the vaporiser system in the period considered. In this way, the output quantity of the vaporised substance can be recorded, particularly in medical applications, with regard to compliance with a desired dosage, and thus easy and reliable monitoring of the dosage can be implemented.

In addition, due to this control option, with regard to using individual doses, it is not necessary to load the vaporiser system individually for each individual dose to be applied or vaporised.

According to a second aspect, the invention also relates to a cartridge for a vaporiser system according to the invention for vaporising a composition, comprising:

at least one reservoir for holding the composition, and

at least one absorber which is adapted to at least partially absorb electromagnetic radiation emitted by an external radiation source and to convert it at least partially into thermal energy and/or to emit it at least partially as electromagnetic radiation with increased wavelength compared to the absorbed electromagnetic radiation,

the absorber being a three-dimensional body, the dimension of which in two spatial directions is greater than or at least equal to the dimension in the third spatial direction, preferably a plate with any base area, in particular a disc, or a cuboid, wherein the absorber preferably has at least one planar or curved surface, preferably at least two, particularly preferably at least four planar surfaces,

the absorber being arranged in the cartridge in such a manner that the composition held in the reservoir is in contact with or can come into contact with the absorber,

the absorber being arranged in the cartridge in such a manner that it can be exposed from outside the cartridge to electromagnetic radiation at the wavelength of which the absorber shows an absorption, preferably an absorption maximum.

Corresponding cartridges according to the invention are suitable for a vaporiser system according to the invention and comprise the absorber identified above as particularly advantageous, which is arranged in the cartridge in such a manner that it can be exposed to electromagnetic radiation from outside the cartridge. The advantages of corresponding cartridges according to the invention emerge from the statements above. The cartridge according to the invention is preferably configured so that it cannot be refilled and/or recycled without processing.

According to a third aspect, the invention further relates to a portable vaporising apparatus comprising a vaporiser system according to the invention for vaporising a composition or a cartridge according to the invention, the first element and the second element being reversibly and detachably connected to each other in a non-destructive manner.

The advantages of corresponding portable vaporising apparatuses according to the invention emerge from the statements above.

According to a fourth aspect, the invention also relates to an absorber for a vaporiser system according to the invention for vaporising a composition, the absorber being adapted to at least partially absorb the electromagnetic radiation emitted by a radiation source and to convert it at least partially into thermal energy and/or to emit it at least partially as electromagnetic radiation with increased wavelength compared to the absorbed electromagnetic radiation,

the absorber being a three-dimensional body, the dimension of which in two spatial directions is greater than or at least equal to the dimension in the third spatial direction, preferably a plate with any base area, in particular a disc, or a cuboid, wherein the absorber preferably has at least one planar or curved surface, preferably at least two, particularly preferably at least four planar surfaces,

the absorber having channels, preferably capillary channels, and/or being a porous solid body such that a passage of the liquid composition is possible through the absorber.

Preferred embodiments of the absorber emerge from incorporating the features referred to above.

In our own experiments, absorbers according to the invention with a corresponding structure have proven not only to be particularly efficient during vaporisation, but are most particularly advantageous for use in vaporiser systems according to the invention, since they can not only function as absorbers, but can also seal off the composition inside the reservoir from the outside world so that it can only penetrate through the absorber from the second element when the latter is heated by exposure to electromagnetic radiation, such that the vaporising composition escapes.

According to a fifth aspect, the invention also relates to a composition for a vaporiser system according to the invention, comprising at least one active ingredient component, at least one first carrier substance boiling higher than the active ingredient component and at least one second carrier substance boiling lower than the active ingredient component, the composition comprising at least one additive which increases the absorptivity of the composition for electromagnetic radiation at a wavelength ranging from 50 μm to 700 nm and/or the composition comprising at least one type of particles, either as a mixture or a dispersion, which is suitable as an absorber material for at least partially absorbing the electromagnetic radiation emitted by a radiation source and converting it at least partially into thermal energy and/or emitting it at least partially as electromagnetic radiation with increased wavelength compared to the absorbed electromagnetic radiation.

Corresponding compositions according to the invention are preferred because it has been shown in comprehensive tests by the inventors that the vaporising behaviour of a composition is particularly advantageous if, in addition to the active ingredient component which has a specific boiling point, there are at least two carrier substances present, the boiling point of which is higher on the one hand and lower on the other hand than that of the active ingredient component. This achieves a vaporising temperature of the composition that is optimum for the active ingredient, while the higher boiling component prevents the system from drying out before the remaining active ingredient vaporises.

The composition can comprise in particular at least one active ingredient component, at least one first carrier substance boiling higher than the active ingredient component and at least one second carrier substance boiling lower than the active ingredient component, the active ingredient component preferably comprising nicotine, tetrahydrocannabinol, cannabidiol or substances of the corresponding substance classes and the composition preferably further comprising one or more solvents selected from the group consisting of 1,2-propanediol, glycerol and water.

The composition according to the invention is specifically adapted to the vaporiser system according to the invention and the method according to the invention and can in particular comprise a dye which increases the absorptivity of the composition in the wavelength range in which the absorber mostly emits the electromagnetic radiation shifted to longer wavelengths. As a result, a composition according to the invention can be vaporised particularly efficiently by the absorber, as the electromagnetic radiation emitted by the absorber is absorbed particularly efficiently. Additionally or alternatively, the composition according to the invention can comprise, as a mixture or dispersion, particles that, as sole or additional absorbers, can assume the function of the absorber in the vaporiser system according to the invention. As explained above, a corresponding composition thus not only has advantages in terms of efficient vaporisation, but also makes it possible to remove the absorber from the second element after use without leaving any residue, for example by rinsing out the remains of the composition.

A composition according to the invention is preferred for use in the treatment of respiratory diseases or in the treatment of pain, the composition preferably being vaporised due to the interaction of the composition with electromagnetic radiation and being inhaled by the patient.

According to a sixth aspect, the invention further relates to a spatial juxtaposition, in particular a kit, of a plurality of components of a vaporiser system or a vaporising apparatus according to the invention, comprising:

A. a first element as a reusable part comprising at least one electrical energy source and connected thereto at least one radiation source which is adapted to emit electromagnetic radiation, and

B. one or more second elements as a disposable part, preferably a cartridge according to the invention, comprising, in at least one reservoir, a composition intended for vaporising and an absorber which is adapted to at least partially absorb the electromagnetic radiation emitted by the radiation source and to convert it at least partially into thermal energy and/or to emit it at least partially as electromagnetic radiation with increased wavelength compared to the absorbed electromagnetic radiation,

the first and the second elements being reversibly and detachably connectable to each other in a non-destructive manner and a radiation conductor being thus arranged in the first and/or second element such that a radiation-conducting connection is formed between the radiation source and the absorber when the first element and the second element are connected to each other.

The spatial juxtaposition of the listed components according to the invention is therefore preferred because vaporiser systems and vaporising apparatuses according to the invention can be stored and transported much more safely and are thus more suitable for sale. Storage and sale in an assembled state always involves the residual risk that the vaporiser system will be unintentionally activated which can be ruled out for the spatial juxtaposition of the components.

Furthermore, it is preferred that the spatial juxtaposition contains a plurality of second elements at once which are available to the user as replacement cartridges as soon as the initial cartridge is empty. In particularly preferred configurations, the second elements contained comprise different compositions, for example liquids with various flavours or compositions with different medicinal active ingredients.

A spatial juxtaposition according to the invention is preferred, additionally comprising a charging device for the electrical energy source,

and/or

additionally comprising operating instructions,

and/or

additionally comprising a refilling device for pouring the composition into a second element,

and/or

a container comprising the composition,

and/or

a data carrier comprising a computer program product which, when executed on a data processing device, causes the device to execute a method for controlling or adjusting a vaporiser system.

According to a seventh aspect, the invention also relates to a method for vaporising a composition in a vaporiser system, comprising the steps:

a) providing a first element comprising at least one radiation source connected to an electrical energy source, which radiation source is adapted to emit electromagnetic radiation,

b) providing a second element comprising at least one reservoir for holding the composition and at least one absorber which is adapted to at least partially absorb the electromagnetic radiation emitted by the radiation source and to convert it at least partially into thermal energy and/or to emit it at least partially as electromagnetic radiation with increased wavelength compared to the absorbed electromagnetic radiation,

c) connecting the first element to the second element such that a radiation-conducting connection is formed between the radiation source and the absorber by a radiation conductor,

d) activating the radiation source and thus vaporising the composition by means of the thermal energy obtained from the absorber due to conversion from the electromagnetic radiation and/or by means of the electromagnetic radiation with increased wavelength compared to the absorbed electromagnetic radiation which is emitted by the absorber.

The method according to the invention is advantageous because it enables controlled and safe vaporisation of a composition, in a vaporiser system which can be operated with a high degree of operational reliability. The method is particularly simple and can also be carried out without specific instruction, even by a user who is not very tech-savvy. Vaporisation in the method according to the invention is also particularly controlled, as activation of the radiation source enables particularly precise input of energy into the composition via the absorber. The method according to the invention preferably comprises, after step d), the step d1) which represents inhalation of the vaporised composition, preferably the nicotine-containing composition.

A method according to the invention is preferred, additionally comprising, after step d), the step:

e) detaching the first and second elements connected to each other,

as well as one or more of the following steps:

f1) providing a further second element and connecting the further second element to the first element for vaporising the composition,

f2) refilling the reservoir in the second element to create a refilled second element and connecting the refilled second element to the first element for vaporising the refilled composition, or

f3) recycling the second element.

A corresponding method is preferred because it is particularly resource-conserving and at the same time enables particularly long-lasting inhalation that is only briefly interrupted. If the need arises, a used cartridge can be replaced directly with an unused cartridge and the vaporiser system can be used again in the method according to the invention. Additionally or alternatively, the reservoir of the used up second element can be refilled. This reduces the need for further cartridges but is regularly perceived as a drawback in terms of operational reliability. It is particularly advantageous to recycle the used-up cartridge.

A method according to the invention is therefore preferred, additionally comprising the step:

h) cleaning the reservoir in the second element, the absorber preferably being removed.

Corresponding methods are therefore particularly advantageous because cleaning of the reservoir in the second element, referred to above, removes residues of foreign components, for example particles of the absorber material, and thus prepares the second element for later recycling.

The advantages mentioned above in connection in particular with the vaporiser system according to the first aspect of the invention also apply correspondingly in principle, to the extent applicable with respect to the particular aspect, to the further aspects of the invention mentioned herein.

Also disclosed is the use of a radiation source, an absorber, preferably an absorber according to the invention, or a composition, preferably a composition according to the invention, in a vaporiser system according to the invention.

As an alternative to the vaporiser system according to the invention, a vaporiser system is disclosed in which the vaporiser system comprises a non-transparent composition, the one or more absorption maxima of which are at a wavelength which is emitted by the radiation source, preferably at a wavelength which is within 20%, preferably within 10%, particularly preferably within 5%, around the intensity maximum of the emission of the radiation source, such that the absorber is formed by the composition, the composition preferably comprising a dye.

### BRIEF DESCRIPTION OF THE DRAWINGS

The invention and preferred embodiments of the invention will be explained and described in greater detail below with reference to the associated drawings. The same reference numbers in different figures denote the same components.

FIG. 1 is a schematic flowchart of the energy and mass transport between the components of a vaporiser system according to the invention;

FIG. 2 is a schematic flowchart of the energy and mass transport between the components of a vaporiser system according to the invention with visualisation of the first and second elements;

FIG. 3 is a schematic cross-section through an exemplary vaporiser system according to the invention;

FIGS. 4a-4c are three schematic representations (4a, 4b, 4c) of exemplary relative arrangements of a radiation source and an absorber with respect to each other;

FIGS. 5a-5c are three schematic cross-sectional representations (5a, 5b, 5c) of exemplary relative arrangements of a radiation source and an absorber with respect to each other in a detail of a vaporiser system according to the invention;

FIG. 6 is a schematic cross-sectional representation of a preferred vaporiser system according to the invention;

FIG. 7 is a detail of a schematic cross-sectional representation of a preferred vaporiser system according to the invention;

FIG. 8 is a detail of a schematic cross-sectional representation of a preferred vaporiser system according to the invention with enlargement of the connection region between the first and the second element;

FIG. 9 is a detail of a schematic cross-sectional representation of a preferred vaporiser system according to the invention;

FIG. 10 is a detail of a schematic cross-sectional representation of a preferred vaporiser system according to the invention;

FIG. 11 is a detail of a schematic cross-sectional representation of a preferred vaporiser system according to the invention;

FIG. 12 is a detail of a schematic cross-sectional representation of a preferred vaporiser system according to the invention with enlargement of the connection region between the first and the second element;

FIG. 13 is a schematic cross-sectional representation of a preferred vaporiser system according to the invention;

FIG. 14 is a schematic cross-sectional representation of a preferred vaporiser system according to the invention;

FIG. 15 is a schematic cross-sectional representation of a preferred vaporiser system according to the invention;

FIG. 16 is a schematic cross-sectional representation of a preferred vaporiser system according to the invention; and

FIG. 17 is a schematic flowchart of the method according to the invention.

### DETAILED DESCRIPTION OF THE INVENTION

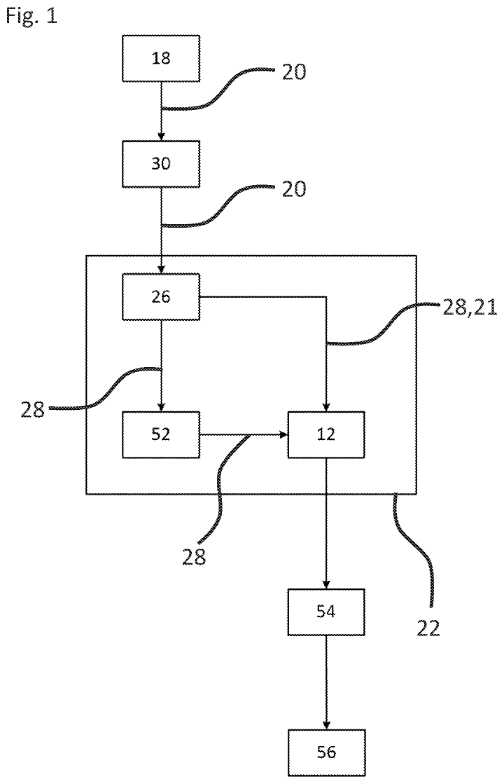

FIG. 1 shows a schematic flowchart of the energy and mass transport between the components of a vaporiser system 10 according to the invention. This flowchart schematically illustrates the function of the vaporiser system according to the invention.

The radiation source 18 emits electromagnetic radiation 20 which impinges on the absorber 26 through the radiation conductor 30, the absorber being adapted to at least partially absorb the electromagnetic radiation 20 emitted by the radiation source 18 and to convert it at least partially into thermal energy 28 and/or to emit it at least partially as electromagnetic radiation 21 with increased wavelength compared to the absorbed electromagnetic radiation 20. In FIG. 1, the absorber 26 is arranged by way of example in the reservoir 24 which is suitable for holding the composition 12.

The thermal energy 28 is supplied to the composition 12 directly or via a circuitous route via a suitable heat conductor 52, the electromagnetic radiation 21 with increased wavelength compared to the absorbed electromagnetic radiation 20 also contributing to the energy input into the composition.

The composition 12 is transferred to the vapour phase in order to produce vapour 54 which can then pass to the user via an outlet opening 56.

In this system, the composition 12 is correspondingly vaporised by means of the thermal energy 28 obtained from the absorber 26 due to conversion from the electromagnetic radiation 20 and/or by means of the electromagnetic radiation 21 with increased wavelength compared to the absorbed electromagnetic radiation 20 which is emitted by the absorber 26.

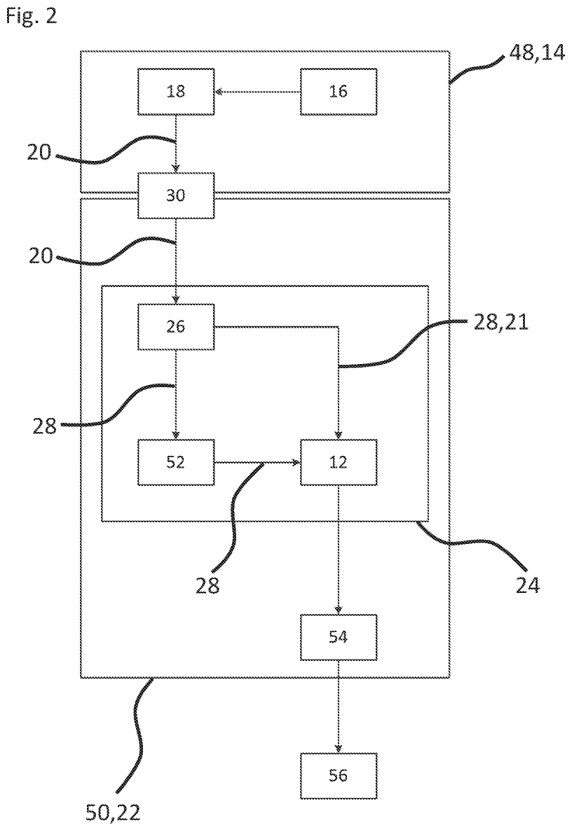

FIG. 2 shows a schematic representation of a vaporiser system according to the invention which is very similar to the representation in FIG. 1. In FIG. 2, however, the first element 14, which is presently configured as reusable part 48, and the second element 22, which is presently configured as disposable part 50, as well as an electrical energy source 16 arranged in the first element 14 and connected to the radiation source 18, are additionally drawn in. Accordingly, it can be seen that the first element 14 comprises at least one radiation source 18 connected to an electrical energy source 16, which radiation source is adapted to emit electromagnetic radiation 20. In addition, the second element 22 comprises a reservoir 24 for holding the composition 12 and the absorber 26.

It is schematically indicated that the radiation conductor 30 is arranged between the first element 14 and the second element 22, it being possible in this case, for example, to configure the radiation conductor in two parts, for example as two transparent glass discs, each of which is arranged in one of the elements and which together form the radiation conductor 30. It can be seen that the radiation conductor 30 is arranged such that a radiation-conducting connection is formed between the radiation source 18 and the absorber 26 when the first element 14 and the second element 22 are connected to each other.

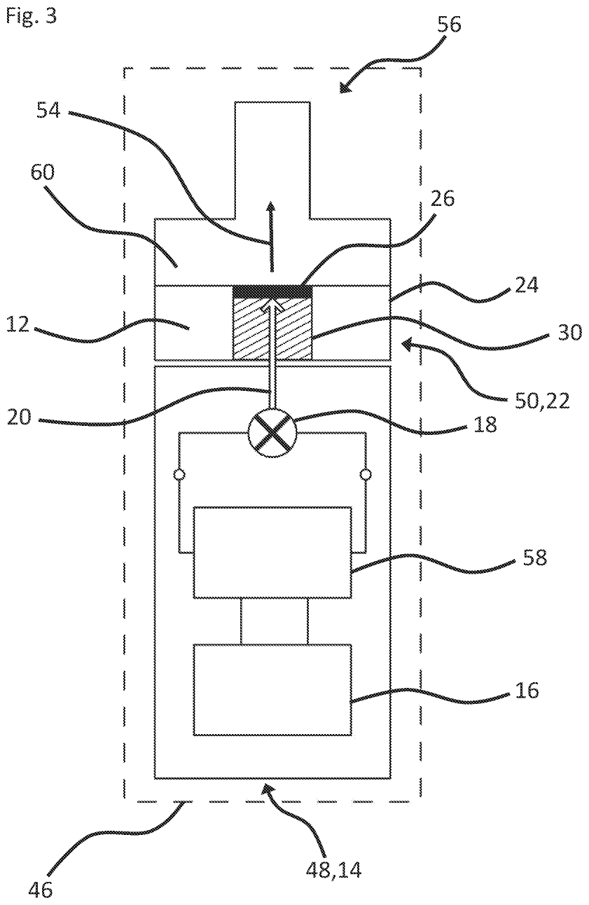

FIG. 3 shows a schematic cross-section through an exemplary vaporiser system 10 according to the invention which is configured as a portable vaporising apparatus 46, for example as an electronic cigarette, which in addition contains the composition 12 as a so-called liquid. The vaporiser system comprises a first element 14, which is configured as reusable part 48, comprising a radiation source 18 connected via a control device 58 to an electrical energy source 16, which radiation source is adapted to emit electromagnetic radiation 20, In addition, the vaporiser system comprises a second element 22, which is configured as disposable part 50, comprising a reservoir 24 with the composition 12 and an absorber 26, which is adapted to at least partially absorb the electromagnetic radiation 20 emitted by the radiation source 18 and to convert it at least partially into thermal energy 28 and/or to emit it at least partially as electromagnetic radiation 21 with increased wavelength compared to the absorbed electromagnetic radiation 20.

The first element 14 and the second element 22 are reversibly and detachably connectable to each other in a non-destructive manner, whereby, in the embodiment shown in FIG. 3, they are reversibly and detachably connected to each other in a non-destructive manner, for example by means of a screw system (not shown). In FIG. 3, the radiation conductor 30 is arranged in the first element 22 in such a manner that a radiation-conducting connection is formed between the radiation source 18 and the absorber 26. In this way, the vaporiser system 10 or the portable vaporising apparatus 46 is adapted to vaporise the composition 12 by means of the thermal energy 28 obtained from the absorber 26 due to conversion from the electromagnetic radiation 20 and/or by means of the electromagnetic radiation 21 with increased wavelength compared to the absorbed electromagnetic radiation 20 which is emitted by the absorber 26.

The radiation source 18 is controlled or regulated by a control device 58. Vaporisation takes place in a vaporising region 60, from which the vapour passes to the outlet opening. Not shown is an inlet for supply air which mixes with the vapour in the vaporising region 60. The radiation source 18 is controlled by the control device 58 so that the portion of the emitted electromagnetic radiation 20 absorbed and converted by the absorber 26 is sufficient to vaporise a defined quantity, for example 6 g, of the composition 12 in 3 s.

In the example shown in FIG. 3, the radiation source 18 is an LED in SMT (surface-mounted technology) construction with a maximum of the emission between 444 and 465 nm, with a typical value of 459 nm and a spectral bandwidth of 27 nm. A vaporiser system according to the invention is preferred, the radiation source being adapted to be operated continuously or pulsed, pulsed operation being preferred.

In the example shown in FIG. 3, the composition 12 is a liquid comprising nicotine as the active ingredient component as well as 1,2-propanediol, glycerol and water. The composition 12 shows almost no absorption at the wavelength of 444 to 465 nm.

In the example shown in FIG. 3, the reservoir 24 is composed of plastic, it also being possible to use other materials.

In the example shown in FIG. 3, the radiation conductor 30 is a cuboid block of quartz glass which is transparent in all spatial directions to the electromagnetic radiation 20 emitted by the radiation source 18, it also being possible, of course, to use other radiation conductors 30.

In the example shown in FIG. 3, the absorber 26 is a copper body configured as a porous three-dimensional body, namely as a plate with 6 planar surfaces, which has been provided with a black coating and which sufficiently absorbs the electromagnetic radiation 20 with a wavelength of 459 nm. Of course, other absorbers can also be used.

In the example shown in FIG. 3, the electrical energy storage device 16 is a lithium-ion battery with a capacity of 650 mAh and a maximum discharge current of 6.5 A, but other electrical storage devices 16 can also be used.

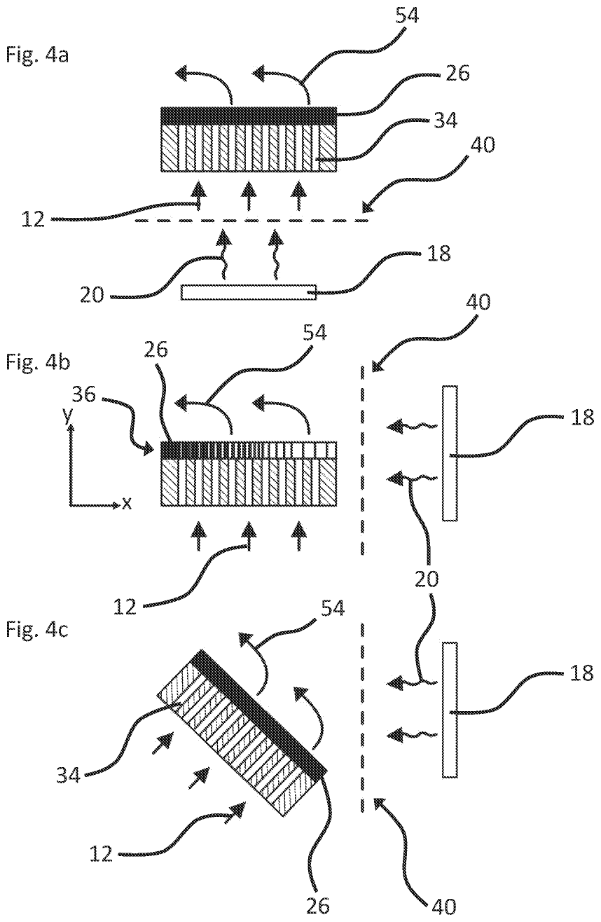

FIGS. 4a to 4c show, in three schematic representations, exemplary relative arrangements of a radiation source 18 and an absorber 26 with respect to each other.

It can be seen in FIG. 4a that the radiation source 18 conducts the electromagnetic radiation 20 through a section of the reservoir 40, i.e. the transparent outer wall, and the composition 12 in such a manner that the radiation impinges vertically, in the Y-direction, on the absorber 26 which has a structure provided with channels 34 on an irradiated surface, through which the composition 12 can be drawn into the absorber 26 by capillary action. In the embodiment shown, the absorber 26 has the greatest absorptivity in the region directed away from the radiation source 18. Vaporisation of the composition 12 and thus the formation of vapour 54 therefore takes place on the side directed away from the radiation source 18. This form of orthogonal irradiation of the absorber 26 has proven particularly successful in terms of efficient energy use.

FIG. 4b shows a setup comparable to FIG. 4a, but this time with the electromagnetic radiation 20 radiating along the X-direction so that it impinges on the narrower side of the absorber 26. However, in this embodiment the absorber 26 has a gradient of absorption along the X-direction which is generated by a concentration gradient of pigments 36, black in this example, with an absorption maximum at the wavelength of the electromagnetic radiation 20 in the silicate glass matrix acting as the absorber in this example.

FIG. 4c shows an arrangement in which the electromagnetic radiation 20 impinges on the absorber 26 at an angle of incidence of approximately 45°, this less efficient arrangement per se always proves its value when more than one absorber 26 is to be used, which are to be activated by the same radiation source.

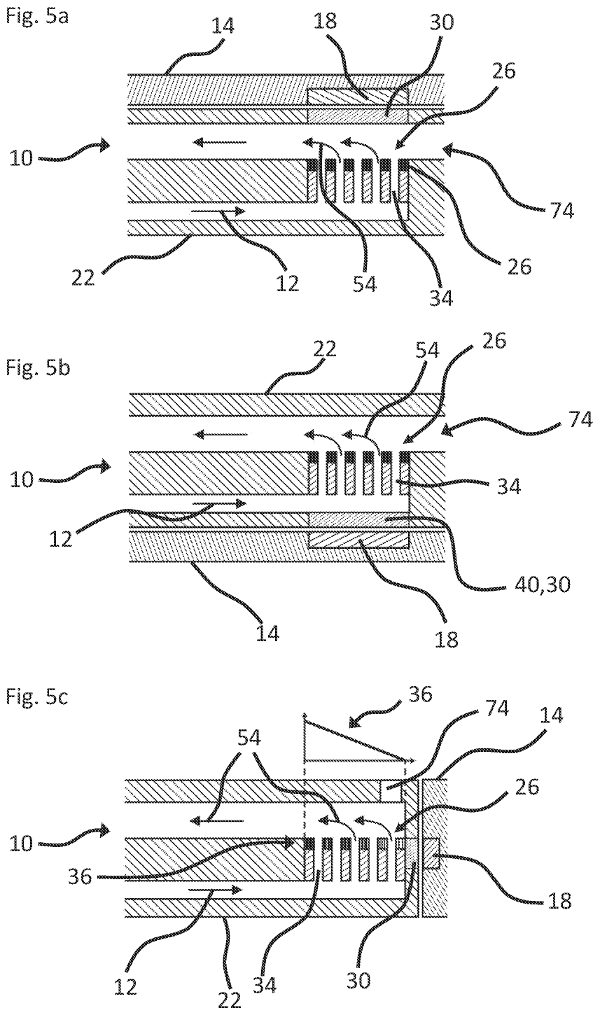

FIGS. 5a to 5c show details of schematically different relative arrangements of the radiation source 18 and the absorber 26 in a vaporiser system 10 according to the invention, as can be implemented structurally by way of example.

In FIGS. 5a to 5c, the first element 14 and the second element 22 in each case are reversibly and detachably connected to each other in a non-destructive manner by fastening means (not shown) such that a radiation-conducting connection, which extends through the radiation conductor 30, is formed between the radiation source 18 and the absorber 26, the radiation conductor 30 in FIG. 5b being configured as a section of the reservoir 40. In all cases shown, the liquid composition 12 passes to the absorber 26 and is transported by capillary forces to the absorbent portion of the absorber 26 (shown in dark) through the channels 34 arranged on this absorber 26, which in these examples are counted as part of the absorber 26, but can also be configured as a porous wick, for example. During operation of the vaporiser system 10, vaporisation of the composition 12 takes place there so that vapour 54 is formed which, together with air provided by a supply air line 74, is conducted to the outlet opening 56 and to the mouthpiece 76 (both not shown). In FIGS. 5a and 5b, the absorber 26 is exposed to electromagnetic radiation 20 from above and below, i.e. once frontally onto the absorbent portion of the absorber 26 and once onto the channels 34. In FIG. 5c, as previously discussed for FIG. 4b, irradiation takes place from the side, a gradient of absorption being generated in turn by a concentration gradient of pigments 36, this gradient being drawn in schematically in FIG. 5c. In this purely schematic representation, the y-axis shows the pigment density in purely qualitative terms which can be an indicator of the maximum absorption, whereas the x-axis shows the location in the absorber 26 and the distance from the radiation source. The schematic representation in FIG. 5c thus shows, by way of example, a linear increase in pigment concentration with increasing distance from the radiation source 18. In other words, it shows a decrease in transparency in the absorbent portion of the absorber 26 as the distance from the radiation source increases along the vapour direction. The person skilled in the art will recognise that the gradient plotted is a purely qualitative representation which, for reasons of clarity, does not take account of the channels 34 in the absorber 26 in which the pigment density is, of course, actually zero. Moreover, in practice, such absorbers 26 have shown the best properties which display a non-linear increase in particle concentration along the x-axis.

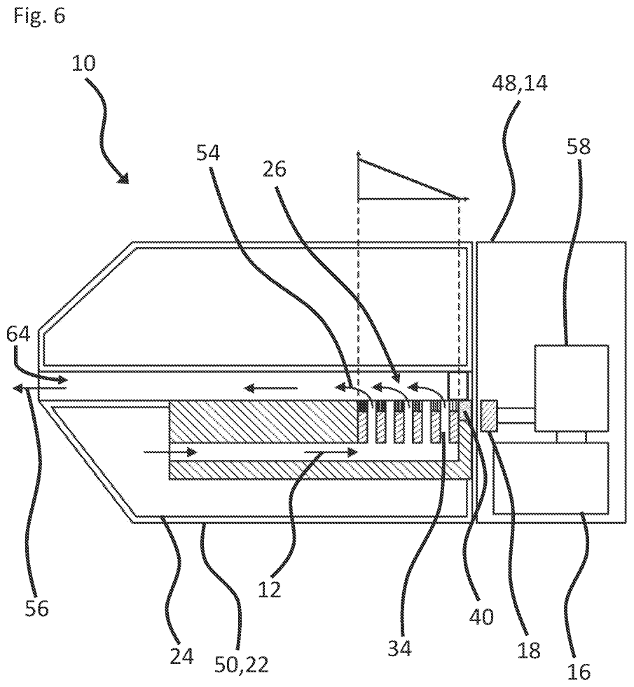

FIG. 6 shows a vaporiser system 10 according to the invention in which the arrangement shown in FIG. 5c is installed. The assembly known from FIG. 5c represents the connection between a reservoir 24 and the composition 12 contained therein as well as the vent 64 which conducts the vapour 54 generated to the outlet opening. In this example, the vent 64 has a circular cross-section and is arranged coaxial with the reservoir 24 which is also circular. These components form the second element 22, or the disposable part 50, which is reversibly and detachably connected to the first element 14, or the reusable part 48, in a non-destructive manner, which houses the electrical energy source 16, the control device 58 and the radiation source 18, the latter irradiating the absorber 26 provided with the absorption gradient laterally through a section of the reservoir 40 which is transparent to the electromagnetic radiation 20.

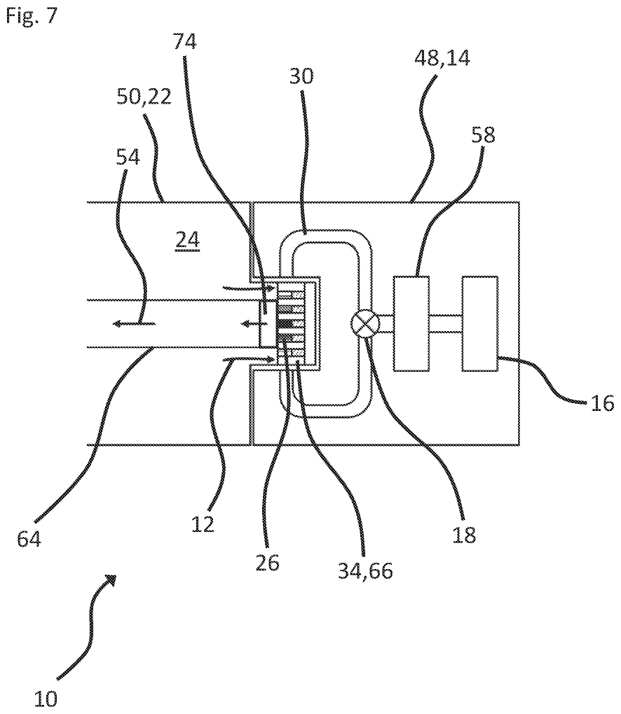

FIG. 7 shows a detail of a preferred embodiment of the vaporiser system 10 according to the invention in cross-section, in which the first element 14 and the second element 22 are connected to each other by means of a positive-locking plug-in connector. The vaporiser system 10 shown is rotationally symmetrical and has a circular cross-section. The radiation conductor 30, in which radiation conduction is based on total or partial reflection, is accordingly arranged around the absorber 26 in an annular shape and thus ensures circumferential irradiation of the absorber 26. The disc-shaped absorber 26, which is provided with channels 34 filled with a wick 66, has a radial, inwardly increasing absorption gradient starting from the edge of the disc, which is irradiated by means of the radiation conductor 30, to the centre of the disc, which gradient is formed by colour particles in an otherwise transparent crystal matrix and thus produces a uniform temperature profile, despite the indirect, lateral irradiation of the absorber 26.

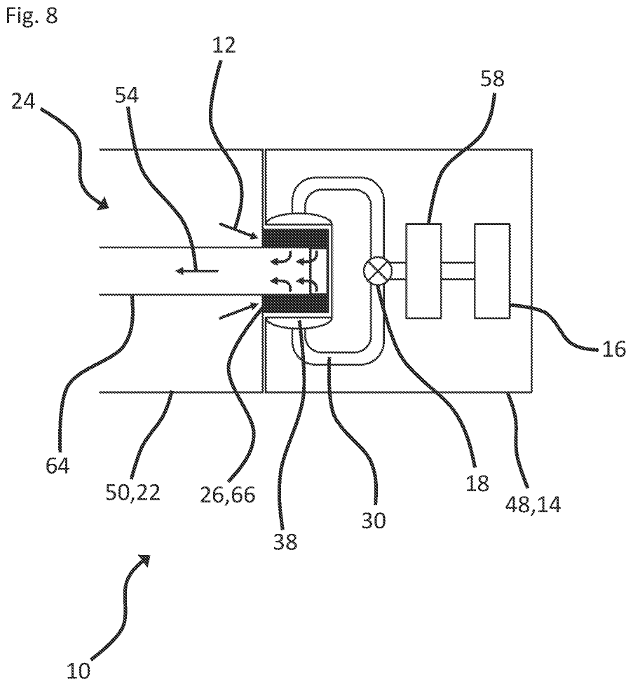

FIG. 8 shows a detail of a preferred embodiment of the vaporiser system 10 according to the invention in cross-section, which is a design modification of FIG. 7, in which the absorber 26 in this case is configured as a porous solid ring of wick material and also serves at the same time as wick 66. The electromagnetic radiation 20 conducted via the radiation conductor 30 is spread by scattering via the radiation former 38 in extension of the radiation conductor 30 so that the entire outer surface of the absorber 26 is impinged on.

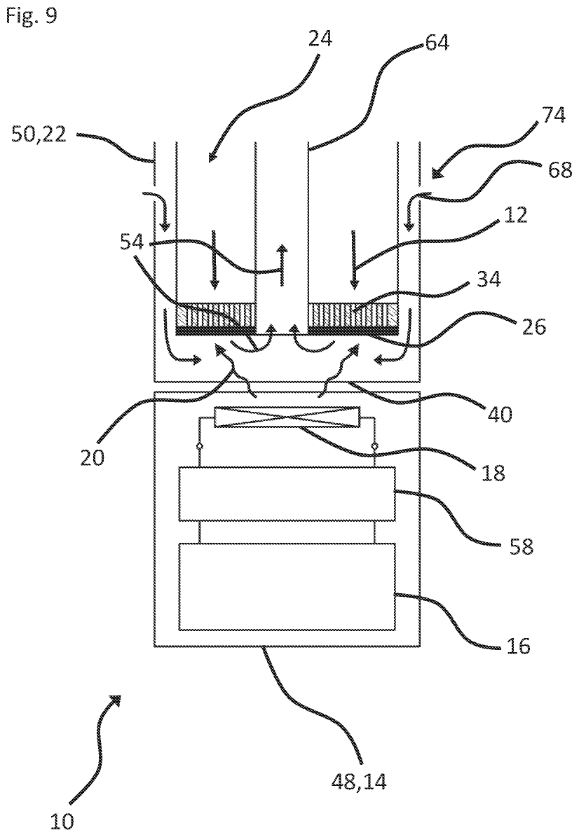

FIG. 9 shows a detail of a preferred embodiment of the vaporiser system 10 according to the invention in cross-section, the individual components which are arranged in this rotationally symmetrical vaporiser system 10 having already been described above. The particularly efficient feature of the embodiment in FIG. 9 is that the annular, disc-like absorber 26 is configured as a porous absorber 26, provided with channels 34, which is formed by the lower base of the reservoir 24. Both the reservoir 24 and the absorber 26 surround the vent which is arranged coaxial with the reservoir 24 and absorber 26. The composition 12 enters the absorber from the reservoir 24 through the channels 34 and is vaporised there due to interaction of the absorber 26 with the electromagnetic radiation 20 as described above. The vapour is entrained by the supply air 68 and leaves the vaporiser system 10 via the vent 64, e.g. in the direction of a user.

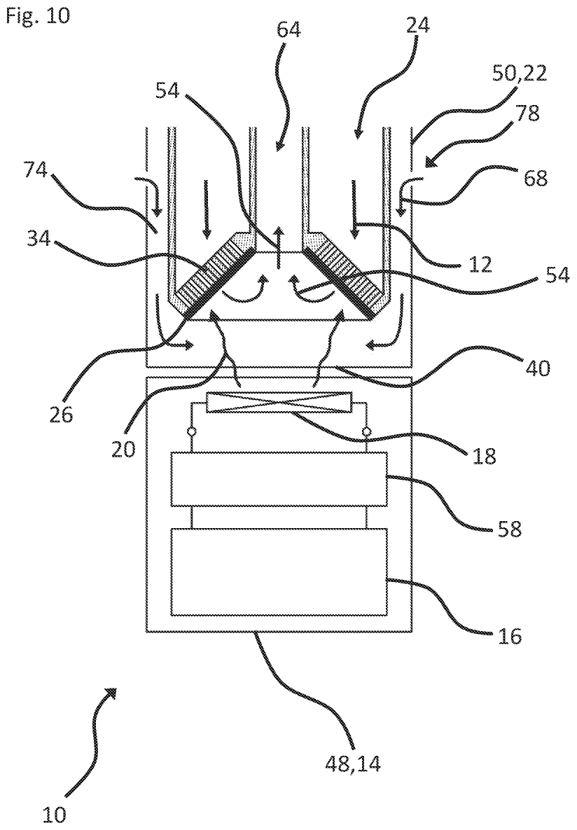

The detail of a preferred embodiment of the vaporiser system 10 according to the invention shown in cross-section in FIG. 10 differs substantially from the representation in FIG. 9 in that a hollow cone-shaped absorber 26 is used instead of an annular absorber 26. This makes it possible to adjust a lower radiation angle at the radiation source 18 due to the inclined positioning relative to a longitudinal axis of the vent 64 and the radiation source 18, while maintaining the same passage surface for the composition 12 and still completely impinging the absorber 26 with electromagnetic radiation. In addition, a smaller structural shape is possible due to positioning of the absorber 26 with respect to the diameter of the reservoir 24 or the second element 22.

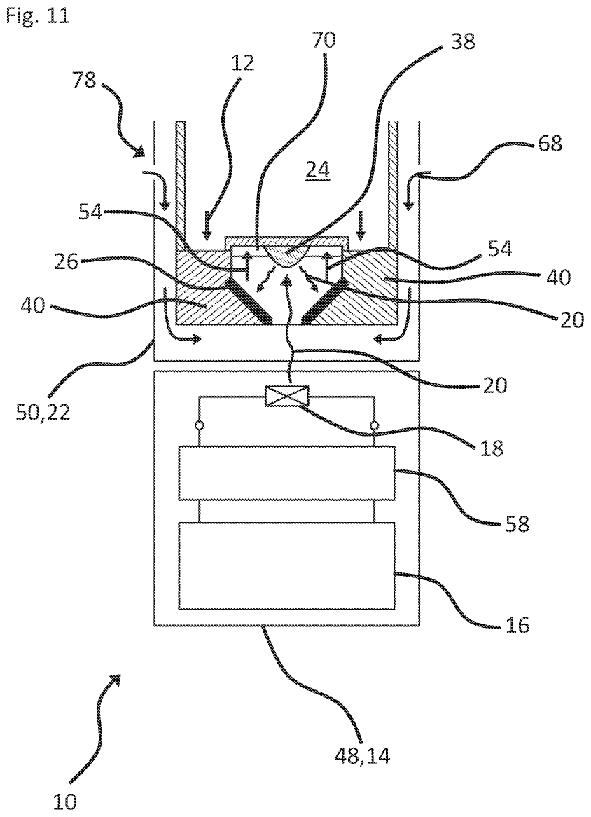

FIG. 11 shows a detail of a preferred embodiment of the vaporiser system 10 according to the invention in cross-section, the composition 12 in the reservoir 24 being supplied to the absorber 26 via an at least partially porous section 40 of the reservoir 24, here the base, it being possible for this section, for example, to also be configured as a separate wick. The absorber 26 is not irradiated by the radiation source 18 in a straight line, but rather the radiation source 18, in the connected state, is aligned with a radiation former 38 which reflects the electromagnetic radiation 20 and redirects it to the absorber 26. From the absorber, the vapour 54 passes to the vent (not shown here) via a connection 70.

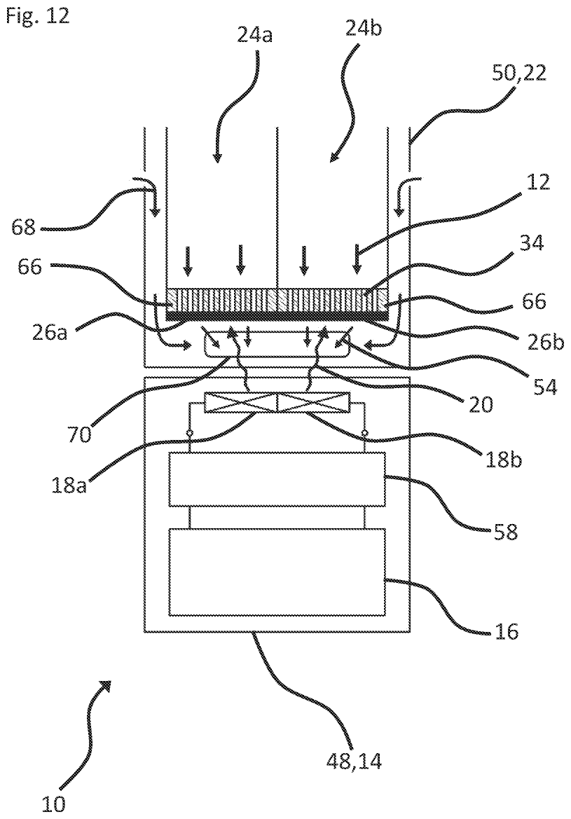

FIG. 12 shows a detail of a preferred embodiment of the vaporiser system 10 according to the invention in cross-section, the vaporiser system 10 comprising two reservoirs 24a and 24b, each of which is connected to one of two absorbers 26a and 26b which can be irradiated with electromagnetic radiation via two separate radiation sources 18a and 18b such that the vapour from the composition 12 can pass from the left and/or right reservoir 24a and 24b to the vent (not shown here) via the connection 70. The vaporiser system 10 accordingly comprises a first absorber 26a and a second absorber 26b as well as a first radiation source 18a and a second radiation source 18b, the first absorber 26a and the second absorber 26b being connected to different, separate sections of the reservoir 24a and 24b.

The embodiment shown functions in principle as described below. The first absorber 26a is supplied with composition 12 by the first reservoir 24a, the first absorber 26a being fluidly coupled to the wick 66 in a liquid-conducting manner and being wetted with the composition 12 by said wick. The same applies to the second absorber 26b. When the vaporiser system 10 is activated, the first radiation source 18a is activated in such a manner that the first radiation source 18a initially illuminates the first absorber surface 26a during the illumination period. During a portion of the illumination period, the absorber 26a absorbs the electromagnetic radiation 20 and converts it (among other things) as described above, e.g. into thermal energy. The composition absorbs the thermal energy and vaporises. After a predetermined time, the first radiation source 18a is deactivated and the second radiation source 18b is activated. The second radiation source 18b illuminates the second absorber 26b as previously described. During a further predetermined time of the illumination period of the second radiation source 18b, composition 12 can flow from the first reservoir 24a into the first absorber 26a. After the predetermined illumination period of the second radiation source 18b, it is switched off. The advantage of this setup is effectively more continuous vaporising of the composition due to the sequential, consecutive illumination of the different absorbers 26a and 26b. As a result, during the period of illumination of the second absorber 26b, the first absorber 26a can be refilled with composition 12 from the corresponding reservoir 24a. Alternatively, in this setup it is also conceivable for the composition 12 in the reservoir 24a and the composition 12 in the reservoir 24b to differ. For example, the reservoir 24a could comprise a composition 12 having nicotine. The reservoir 24b could comprise a composition containing cannabidiol or tetrahydrocannabinol. The radiation sources 18a and 18b can then be operated independently of each other, i.e. for example, according to selection of the desired active ingredient by the user. A further example of two such compositions which differ from each other in the reservoir 24a and 24b can be active ingredients that are used in the therapy of respiratory diseases. For this purpose, the reservoir 24a can comprise a composition 12 having an active ingredient which a patient takes regularly according to a schedule determined by a doctor. The reservoir 24b can comprise an active ingredient which the patient can use in an emergency. In this case too, operation of the radiation sources 18a, 18b would depend on selection by the patient of the active ingredient to be vaporised in the reservoir 24a and 24b respectively.

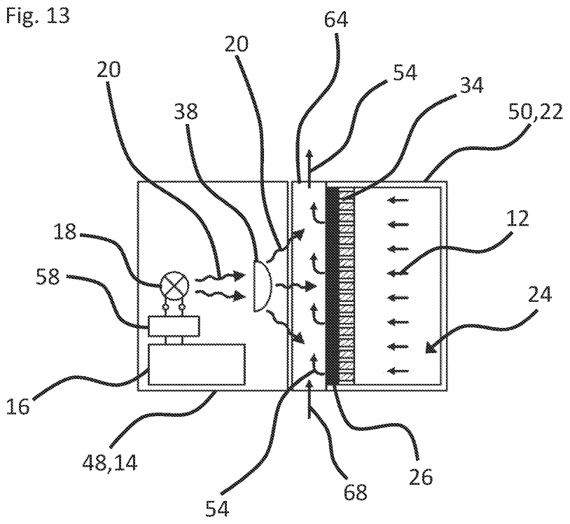

FIG. 13 shows a preferred embodiment of the vaporiser system 10 according to the invention in cross-section, this being a stationary setup such as can be used in inhalers. Of particular note here is that a radiation former 38 is used to scatter the relatively focused electromagnetic radiation 20 of a monochromatic laser radiation source 18 such that a relatively large surface of the absorber 26 can be impinged on, in order to enable uniform vaporising of the composition 12, even with a laser.

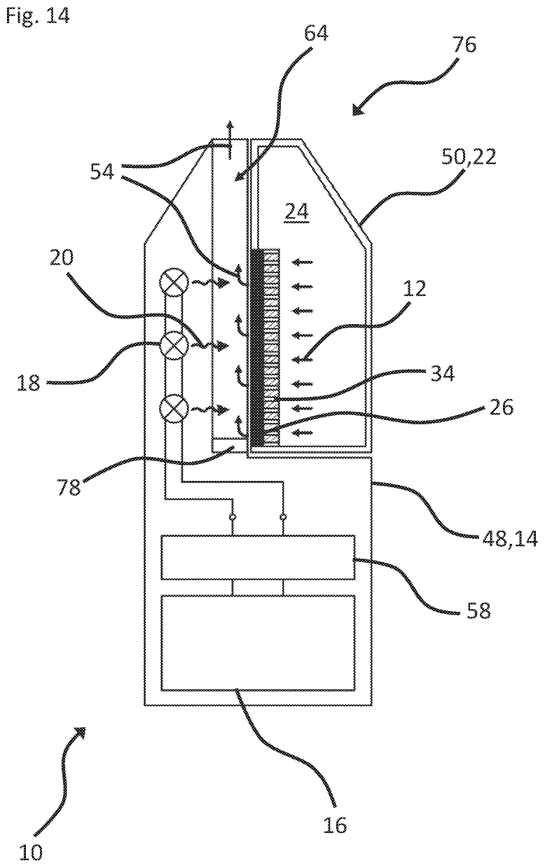

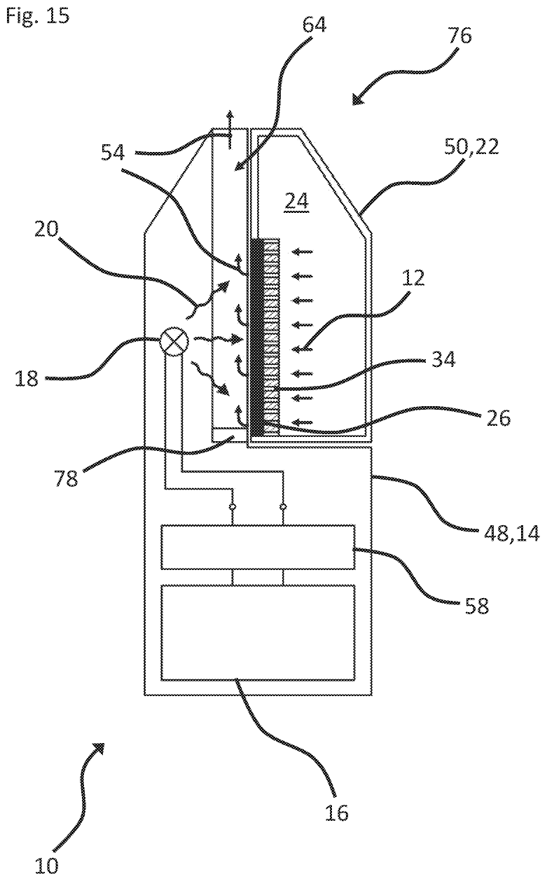

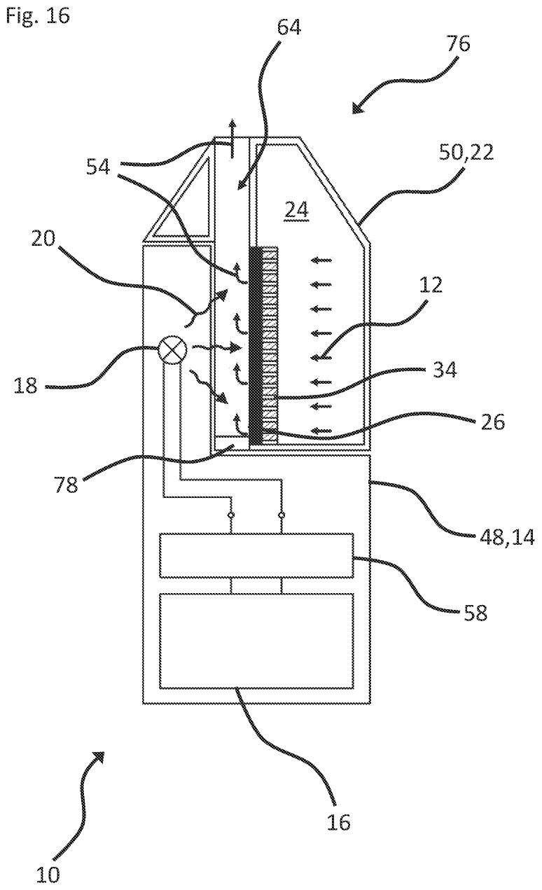

FIGS. 14, 15 and 16 show particularly preferred embodiments of the vaporiser system 10 according to the invention in cross-section, the second element 22 being configured in each case as mouthpiece 76 (FIG. 16), or forming a mouthpiece 76 together with the first element 14 (FIGS. 14 and 15). FIG. 14 shows a particularly powerful vaporiser system 10 which enables particularly intensive and uniform vaporisation via the total of three radiation sources 18. In FIGS. 14 and 15, the vent 64, which conducts the vapour 54 to the mouthpiece 76, is formed between the first element 14 and the second element 22 which is made possible in that the absorber 26 prevents undesirable leakage of the composition 12 from the reservoir 24. In contrast, the vent 64 in the embodiment shown in FIG. 16 is integrated into the cartridge, which is most preferred, since the reusable part 48 does not come into contact with the composition even if unintentional leakage of the composition 12 from the reservoir 24 occurs, e.g. due to mechanical damage to the absorber 26.

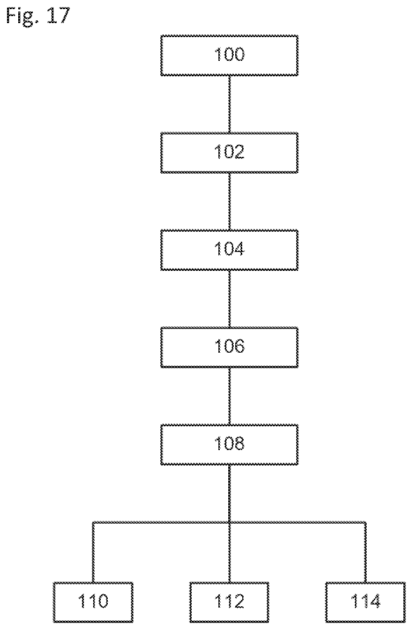

FIG. 17 shows a schematic flowchart of the method according to the invention, which comprises the steps illustrated, namely:

providing 100 a first element 14 comprising at least one radiation source 18 connected to an electrical energy source 16, which radiation source is adapted to emit electromagnetic radiation 20,

providing 102 a second element 22 comprising at least one reservoir 24 for holding the composition 12 and at least one absorber 26 which is adapted to at least partially absorb the electromagnetic radiation 20 emitted by the radiation source 18 and to convert it at least partially into thermal energy 28 and/or to emit it at least partially as electromagnetic radiation 21 with increased wavelength compared to the absorbed electromagnetic radiation 20,

connecting 104 the first element 14 with the second element 22 such that a radiation-conducting connection is formed between the radiation source 18 and the absorber 26 by a radiation conductor 30, and

activating 106 the radiation source 18 and thus vaporising the composition 12 by means of the thermal energy 28 obtained from the absorber 26 due to conversion from the electromagnetic radiation 20 and/or by means of the electromagnetic radiation 21 with increased wavelength compared to the absorbed electromagnetic radiation 20 which is emitted by the absorber 26.

Also shown are the optional steps of the preferred method 108, 110, 112 and 114, namely: detaching 108 the first 14 and second elements 22 connected to each other; providing 110 a further second element 22 and connecting the further second element 22 to the first element 14 for vaporising the composition 12; refilling 112 the reservoir 24 in the second element 22 to create a refilled second element 22 and connecting the refilled second element 22 to the first element 14 for vaporising the refilled composition 12; or recycling 114 the second element 22.

### REFERENCE NUMBERS

10 Vaporiser system

12 Composition

14 First element

16 Electrical energy source

18 Radiation source

18a First radiation source

18b Second radiation source

20 Electromagnetic radiation

21 Electromagnetic radiation with increased wavelength

22 Second element

24 Reservoir

24a First separate section of reservoir

24b Second separate section of reservoir

26 Absorber

26a First absorber

26b Second absorber

28 Thermal energy

30 Radiation conductor

32 Planar or curved surface

34 Channels

36 Concentration gradient of pigments

38 Radiation former

40 Section of reservoir

42 Different, separate sections of reservoir

44 Cartridge

46 Portable vaporising apparatus

48 Reusable part

50 Disposable part

52 Heat conductor

54 Vapour

56 Outlet opening

58 Control device

60 Vaporising region

62 Wall (optional)

64 Vent

66 Wick

68 Supply air

70 Connection to vent

72 Connection to reservoir

74 Supply air line

76 Mouthpiece

78 Supply air inlet

100 Providing a first element

102 Providing a second element

104 Connecting the first element to the second element

106 Activating the radiation source and thus vaporising

108 Detaching the first and second elements connected to each other

110 Providing a further second element and connecting the further second element

112 Refilling the reservoir in the second element

114 Recycling the second element

X, Y, Z Spatial direction

1. A vaporiser system for vaporising a composition, comprising:
a first element comprising at least one radiation source connected to an electrical energy source, which at least one radiation source is adapted to emit electromagnetic radiation; and
a second element comprising at least one reservoir for holding the composition and at least one absorber which is adapted to at least partially absorb the electromagnetic radiation emitted by the at least one radiation source and to convert the electromagnetic radiation at least partially into thermal energy and/or to emit the electromagnetic radiation at least partially as electromagnetic radiation with increased wavelength compared to the absorbed electromagnetic radiation;
wherein the first and the second element are reversibly and detachably connectable to each other in a non-destructive manner and wherein a radiation conductor is arranged such that a radiation-conducting connection is formed between the at least one radiation source and the at least one absorber when the first element and the second element are connected to each other; and
wherein the vaporiser system is adapted to vaporise the composition by means of the thermal energy obtained from the at least one absorber due to conversion from the electromagnetic radiation and/or by the electromagnetic radiation with increased wavelength compared to the absorbed electromagnetic radiation which is emitted by the at least one absorber.

2. The vaporiser system according to claim 1, wherein the emitted electromagnetic radiation has the highest intensity maximum below a wavelength of 500 nm, or ranging from 410 to 490 nm, or 430 to 480 nm, or 440 to 470 nm, wherein the electromagnetic radiation has a spectral bandwidth at 50% of the maximum intensity of 5 to 50 nm, or 10 to 40 nm, or 20 to 30 nm.

3. The vaporiser system according to claim 1, wherein:
the at least one absorber is a three-dimensional body, a dimension of which in two spatial directions is greater than or at least equal to a dimension in a third spatial direction; or
the vaporiser system comprises a composition and the absorber is formed by particles which are mixed with the composition to be vaporised or dispersed in the composition to be vaporised.

4. The vaporiser system according to claim 1, wherein the absorber is configured such that one or more of its absorption maxima for electromagnetic radiation lie at a wavelength of the electromagnetic radiation which is emitted by the at least one radiation source at a wavelength that lies within 20% around an intensity maximum of the emission of the at least one radiation source.

5. The vaporiser system according to claim 1, wherein the absorber has channels or capillary channels, and/or is a porous solid body such that the absorber is liquid-conducting and passage of the liquid composition through the at least one absorber is possible, wherein the at least one absorber comprises a membrane which only allows passage of the liquid composition through the absorber when a limit temperature is exceeded.

6. The vaporiser system according to claim 1, wherein the at least one absorber has a non-homogeneous absorption behaviour along at least one spatial direction and a gradient of absorption along a spatial direction corresponding to a direction of incidence of the electromagnetic radiation onto the at least one absorber.

7. The vaporiser system according to claim 1, wherein the at least one radiation source is a lamp, a laser or a light-emitting diode.

8. The vaporiser system according to claim 1, wherein the radiation conductor is opaque to electromagnetic radiation with a wavelength which deviates more than 50%, from a wavelength of an intensity maximum of the electromagnetic radiation emitted by the at least one radiation source.

9. The vaporiser system according to claim 1, wherein the absorber has at least one planar surface, and wherein the at least one radiation source, the radiation conductor, any radiation formers that may be present and the at least one absorber, when the first and second elements are connected to each other, are arranged in such a way that the electromagnetic radiation impinges on one of the planar surfaces of the absorber at an angle of incidence of less than 45°.

10. The vaporiser system according to claim 1, wherein the reservoir is transparent at least in one section, or transparent to visible light, or transparent to electromagnetic radiation, the wavelength of which is within 20%, around an intensity maximum of an emission of the at least one radiation source.

11. The vaporiser system according to claim 1, wherein the at least one absorber comprises a first absorber and a second absorber and the at least one radiation source comprises a first radiation source and a second radiation source, wherein the first and the second absorber are connected to different, separate sections of the reservoir and the first and the second radiation source have their highest emission maximum at different wavelengths, wherein the absorptivity of the two absorbers differs at at least one of the wavelengths of the highest emission maximum of the two radiation sources by more than 50%.

12. A cartridge for the vaporiser system for vaporising a composition according to claim 1, wherein the cartridge comprises:
the at least one reservoir for holding the composition; and
the at least one absorber which is adapted to at least partially absorb electromagnetic radiation emitted by an external radiation source and to convert the electromagnetic radiation at least partially into thermal energy and/or to emit the electromagnetic radiation at least partially as electromagnetic radiation with increased wavelength compared to the absorbed electromagnetic radiation;
wherein the at least one absorber is a three-dimensional body, a dimension of which in two spatial directions is greater than or at least equal to a dimension in a third spatial direction wherein the absorber has at least one planar or curved surface,
wherein the at least one absorber is arranged in the cartridge in such a manner that the composition held in the reservoir is in contact with or can come into contact with the at least one absorber,
wherein the at least one absorber is arranged in the cartridge in such a manner that the at least one absorber is exposed from outside the cartridge to electromagnetic radiation at a wavelength of which the at least one absorber shows an absorption.

13. A portable vaporising apparatus comprising the vaporiser system for vaporising a composition according to claim 1, wherein the first element and the second element are reversibly and detachably connected to each other in a non-destructive manner.

14. An absorber for the vaporiser system for vaporising a composition according to claim 1, wherein the absorber is adapted to at least partially absorb electromagnetic radiation emitted by a radiation source and to convert the electromagnetic radiation at least partially into thermal energy and/or to emit the electromagnetic radiation at least partially as electromagnetic radiation with increased wavelength compared to the absorbed electromagnetic radiation;
wherein the absorber is a three-dimensional body, the dimension of which in two spatial directions is greater than or at least equal to the dimension in a third spatial direction, wherein the absorber has at least one planar or curved surface;
wherein the absorber has channels or capillary channels, and/or is a porous solid body such that a passage of the liquid composition is possible through the absorber.

15. A composition for the vaporiser system according to claim 1, comprising:
at least one active ingredient component;
at least one first carrier substance boiling higher than the active ingredient component;
at least one second carrier substance boiling lower than the active ingredient component;
wherein the composition further comprises;
at least one additive which increases an absorptivity of the composition for electromagnetic radiation at a wavelength ranging from 50 μm to 700 nm; and/or
at least one type of particles, either as a mixture or a dispersion, which is suitable as an absorber material for at least partially absorbing electromagnetic radiation emitted by a radiation source and converting the electromagnetic radiation at least partially into thermal energy and/or emitting the electromagnetic radiation at least partially as electromagnetic radiation with increased wavelength compared to the absorbed electromagnetic radiation.

16. A spatial juxtaposition of a plurality of components of the vaporiser system according to claim 1, comprising:
A. a first element as a reusable part comprising the electrical energy source and connected thereto the at least one radiation source which is adapted to emit electromagnetic radiation, and
B. one or more second elements as a disposable part, preferably a cartridge, comprising, in at least one reservoir, a composition intended for vaporising and an absorber which is adapted to at least partially absorb the electromagnetic radiation emitted by the radiation source and to convert the electromagnetic radiation at least partially into thermal energy and/or to emit the electromagnetic radiation at least partially as electromagnetic radiation with increased wavelength compared to the absorbed electromagnetic radiation,
the cartridge comprising;
the at least one reservoir for holding the composition; and
the at least one absorber which is adapted to at least partially absorb electromagnetic radiation emitted by an external radiation source and to convert the electromagnetic radiation at least partially into thermal energy and/or to emit the electromagnetic radiation at least partially as electromagnetic radiation with increased wavelength compared to the absorbed electromagnetic radiation;
wherein the at least one absorber is a three-dimensional body, a dimension of which in two spatial directions is greater than or at least equal to a dimension in a third spatial direction wherein the absorber has at least one planar or curved surface;
wherein the at least one absorber is arranged in the cartridge in such a manner that the composition held in the reservoir is in contact with or can come into contact with the at least one absorber;
wherein the at least one absorber is arranged in the cartridge in such a manner that the at least one absorber is exposed from outside the cartridge to electromagnetic radiation at a wavelength of which the at least one absorber shows an absorption;
wherein the first and the second elements are reversibly and detachably connectable to each other in a non-destructive manner and wherein a radiation conductor is arranged in the first and/or second element such that a radiation-conducting connection is formed between the radiation source and the absorber when the first element and the second element are connected to each other.

17. A method for vaporising a composition in a vaporiser system, comprising the steps:
a) providing a first element comprising at least one radiation source connected to an electrical energy source which is adapted to emit electromagnetic radiation;
b) providing a second element comprising at least one reservoir for holding the composition and at least one absorber which is adapted to at least partially absorb the electromagnetic radiation emitted by the radiation source and to convert the electromagnetic radiation at least partially into thermal energy and/or to emit the electromagnetic radiation at least partially as electromagnetic radiation with increased wavelength compared to the absorbed electromagnetic radiation;
c) connecting the first element with the second element such that a radiation-conducting connection is formed between the radiation source and the absorber by a radiation conductor; and
d) activating the radiation source and thus vaporising the composition by the thermal energy obtained from the absorber due to conversion from the electromagnetic radiation and/or by the electromagnetic radiation with increased wavelength compared to the absorbed electromagnetic radiation which is emitted by the absorber.

18. The method according to claim 17, further comprising, after step d), the step:
e) detaching the first and second elements connected to each other;
as well as one of the following steps:
f1) providing a further second element and connecting the further second element to the first element for vaporising the composition;
f2) refilling the reservoir in the second element to create a refilled second element and connecting the refilled second element to the first element for vaporising the refilled composition; or
f3) recycling the second element.

19. The vaporiser system according to claim 6, wherein the gradient of absorption is generated by a concentration gradient of pigments having an absorption maximum at the wavelength of the electromagnetic radiation in the absorber which is otherwise or largely transparent at this wavelength.
